(19) United States
(12) Patent Application Publication
(10) Pub. No.: US 2010/0069983 A1
(43) Pub. Date: Mar. 18, 2010

(54) TRANSMUSCULAR LEFT VENTRICULAR CARDIAC STIMULATION LEADS AND RELATED SYSTEMS AND METHODS

(75) Inventors: James C. Peacock, III, San Carlos, CA (US); Jonathan Langberg, Atlanta, GA (US); Sean Patrick Whalen, Nashville, TN (US)

Correspondence Address: James C. Peacock III, 3317 Melendy Drive, San Carlos, CA, 94070, US

(73) Assignee: EMERGE MEDSYSTEMS, LLC, San Carlos, CA (US)

(21) Appl. No.: 12/436,968

(22) Filed: May 7, 2009

Related U.S. Application Data
(63) Continuation of application No. PCT/US2007/084184, filed on Nov. 8, 2007.
(60) Provisional application No. 60/864,971, filed on Nov. 8, 2006.

(51) Int. Cl. A61N 1/05; A61N 1/362

(52) U.S. Cl. 607/9; 607/127

(57) ABSTRACT

A cardiac stimulation system and method delivers a left ventricle stimulator from a right ventricle lead system in the right ventricle chamber, into a right side of an interventricular septum at a first location, and transmuscularly from the first location to a second location along the left side of the septum. The left ventricle stimulator is affixed at the second location for transmuscular stimulation of the left ventricle conduction system. A biventricular stimulation system further includes a right ventricle stimulator also delivered by the right ventricle lead system to the first location along the right side of the septum for right ventricular stimulation. An energy source is coupled to the transmuscular stimulation system, i.e., a pacemaker, and/or defibrillator, or to enhance contractility, and may be coupled directly or via “leadless” system(s). Various highly beneficial particular arrangements of stimulators and leads are further described.

### CROSS-REFERENCE TO RELATED APPLICATIONS

This application claims priority from, and is a 35 U.S.C. §111(a) continuation of, co-pending PCT international application serial number PCT/US2007/084184, filed on Nov. 8, 2007, incorporated herein by reference in its entirety, which designates the U.S., and which claims priority from U.S. Provisional Patent Application Ser. No. 60/864,971, filed on Nov. 8, 2006, incorporated herein by reference in its entirety. Priority is claimed to each of these applications.

This application is also related to PCT International Publication No. WO 2008/058265 published on May 15, 2008, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

The present invention is a system and method for stimulating the heart.

More specifically, it is a system and method for stimulating the left ventricle, such as for biventricular pacing.

2. Description of Related Art

Many systems and methods have been described for pacing, defibrillating, and/or enhancing contractility of the heart. To do so for the left ventricle poses particular challenges.

In the setting of biventricular pacing where both right and left ventricles are paced, multiple lead systems are used. This typically includes an atrial lead, a right ventricle (RV) lead, and a left ventricle (LV) lead. As for the left ventricle lead approach in these systems, the prevailing approach provides an electrode via a typically guide-wire tracking, valved lead into the coronary sinus system for transvascular placement to initiate contraction of the left ventricle. A separate right ventricle lead is used via the right hear chambers and affixed into the RV endocardium there, which may be along the septal wall.

Despite their role as the prevailing approach for LV stimulation, coronary sinus-based delivery of LV leads is wrought with challenges and shortcomings. The delivery itself can be tedious, and often it is not possible to position the electrode properly for acceptable results. Often seemingly acceptable positioning can displace under physiologic motion in the beating heart system. Once positioned, the stimulated LV contraction may be non-physiological because it is initiated on the epicardial surface of the LV free wall, remove from the specialized conduction system. Non-physiological, underperforming contraction can result with sub-optimal LV ejection profiles. Such issues may adversely effect the efficiency and outcomes of conventional biventricular pacing implant procedures.

A need still exists for improved LV stimulation lead systems.

A need still exists for improved biventricular stimulation systems.

A need still exists for an improved LV stimulation lead system that is easier and more efficient to implant.

A need still exists for an improved LV stimulation lead system that provides more physiologic results in LV hemodynamic function.

A need still exists for an improved LV stimulation lead system that stimulates both the RV and the LV via a single lead assembly.

### BRIEF SUMMARY OF THE INVENTION

One aspect of the present disclosure is a biventricular stimulation lead system that includes a single lead with two electrodes that are adapted to stimulate the right ventricle and left ventricle, respectively, via the interventricular septum.

Another aspect is a left ventricle (LV) stimulation lead that is delivered transmuscularly across the interventricular septum to a left side thereof so as to stimulate the left bundle aspects of contractile conduction coupled to the left ventricle.

Another aspect is a biventricular stimulation lead system with a lead member and dual electrodes that include a first right sided electrode coupled to the distal end of the lead member and that is adapted to be secured to a right side of an interventricular septum in a patient's heart, and a second left sided electrode adapted to be extended from the lead member and transmuscularly to a left side of the septum. Each of the two electrodes is adapted to be coupled to a stimulation source. According to this aspect, the right and left ventricles of the heart are configured to be stimulated by the first and second electrodes, respectively.

Another aspect is a tissue stimulation system with an elongated body with a proximal end portion and a distal end portion with a distal tip; a first active fixation tissue stimulator coupled to the distal end portion at the distal tip; an inner passageway through the first active fixation tissue stimulator; and a second active fixation tissue stimulator positioned within the passageway of the first active fixation tissue stimulator. The first active fixation tissue stimulator is configured to be actively affixed to a first location along a first side of a body space wall upon distal advancement of the first active fixation stimulator against the first side. The second stimulator is extendable distally from the passageway and through the body space wall to an extended location that is distal from the first location and through a second side of the body space wall opposite the first side. The second stimulator is configured to be actively affixed to a second location along the second side of the body space wall upon proximal withdrawal from the extended location to the second location that is more proximally located relative to the first location than the extended location.

Another aspect is a cardiac stimulation system with a right ventricle (RV) stimulator configured to be affixed at a first location along a right side of an interventricular septum; a left ventricle (LV) stimulator configured to be affixed at a second location to a left side of the septum; and a member cooperating with the RV and LV stimulators and configured to extend between the RV and LV stimulators at the first and second locations, respectively.

Another aspect of the present disclosure is a cardiac stimulation system, comprising a right ventricle (RV) stimulator configured to be affixed at a first location along a right side of an interventricular septum; and a left ventricle (LV) stimulator configured to be positioned transmuscularly from the right ventricle chamber, across an interventricular septum, and to be affixed at a second location along a left side of the septum.

According to one mode of certain aspects, a right ventricle stimulator configured to be affixed to a first location along a right side of the septum; and wherein the location of the left ventricle stimulator is a second location.

According to one other mode of the present aspects, an energy source is configured to be coupled to and energize the RV and LV stimulators sufficient to stimulate the right and left ventricles, respectively.

In another mode, an energy source is configured to be coupled to and energize the RV and LV stimulators sufficient to stimulate the right and left ventricles, respectively.

In another mode, the LV stimulator is extendable from the RV stimulator affixed at the first location along the right side of the septum to as to be advanced transmuscularly to the second location along the left side of the septum. In one embodiment of this mode, the RV stimulator comprises a lumen and the second stimulator is advanceable through and from the lumen toward the second location. In another embodiment, the RV stimulator comprises a coiled conductive electrode filament as an active fixation screw electrode, and the LV stimulator comprises an extendable electrode housed within and extendable from the lumen formed within an internal passageway of the coil.

In still a further embodiment, a pusher is coupled to the extendable electrode and configured to push the extendable electrode from the lumen and transmuscularly across the septum to the second location. In further aspects of this embodiment, the pusher includes a pointed needle tip with a sensing electrode and is configured to puncture across the septum under an applied push force and to sense electrical signals at the second location. According to further variation of this aspect, the extendable electrode comprises a through lumen with an inner diameter and the pusher comprises a distal end portion with a first outer diameter and a proximal end portion with a second outer diameter that is larger than the first outer diameter. In addition, the first outer diameter is smaller than the inner diameter, the second outer diameter is larger than the inner diameter, and the pusher is configured to advance its distal end portion distally through the through lumen and to push the extendable electrode distally upon confronting engagement between the extendable electrode and the proximal end portion of the pusher that does not fit within the through lumen.

In another pusher embodiment, an electrical insulator extends over and covering the pusher proximally of the pointed needle tip.

In still another pusher embodiment, the pusher is removable by proximal withdrawal after advancing the extendable electrode to the second location.

In a further embodiment of various energy source modes of the present aspects, the energy source is implantable. In another embodiment, the energy source is coupled to the stimulators via RV and LV coupling members, respectively. In another embodiment, the energy source is configured to be coupled to and energize the stimulators from a remote location via energy delivered across tissue to the stimulators and converted by the stimulators to stimulate tissue at the first and second locations, respectively, such that the energy source and stimulators together comprise a leadless cardiac stimulation system. In still another embodiment, the energy source is a pacemaker. In still another, it is a defibrillator. In yet still another, the energy source comprises a source adapted to enhance contractility of at least the left ventricle via the left ventricle stimulator.

According to a further mode of the present aspects, a stimulation system includes an RV lead with a body with a proximal end portion and a distal end portion. The RV stimulator is coupled to the distal end portion of the lead member and configured to be affixed to the right side of the interventricular septum by distal advancement of the distal end portion against the right side. In addition, the LV stimulator is configured to be extended from the distal end portion of the lead member and to be extended transmuscularly across the septum and to be affixed to a left side of the septum.

According to one embodiment of this mode, the RV lead is configured to couple the first and second stimulators at the first and second locations, respectively, to an energy source at least in part across the right ventricle and right atrium.

In a further variation of this embodiment, the RV lead further comprises a proximal RV coupling member extending proximally from the proximal end portion of the lead body and configured with a proximal RV electrical coupler; and a proximal LV coupling member extending proximally from the proximal end portion of the lead body and configured with a proximal LV electrical coupler. The proximal RV electrical coupler is electrically coupled to the RV stimulator via an RV conductor extending therebetween along the proximal RV coupling member and the lead body, and is adapted to be coupled to an RV stimulation coupler of a cardiac stimulation actuator. The proximal LV electrical coupler is electrically coupled to the LV stimulator via an LV conductor extending therebetween along the proximal LV coupling member and lead body, and is adapted to be coupled to an LV stimulation coupler of a cardiac stimulation actuator. In one further feature of this variation, the lead body comprises a passageway extending between a proximal port along its proximal end portion and a distal port along the distal end portion and communicating distally through a lumen of the RV electrode; the proximal LV coupling member is slideably engaged within the passageway through proximal port, with the LV conductor extending along the passageway and through the distal port to the LV stimulator; and the proximal RV coupling member comprises a substantially fixed extension of the proximal end portion of the lead body. In still another further variation, at least one hemostatic valve located within the passageway and allowing slideable engagement of the LV conductor therethrough without substantial fluid ingress into the passageway across the valve. This may comprises a distal valve located adjacent to or at the distal port, or a proximal valve located adjacent to or at the proximal port, or both.

According to another RV lead embodiment, the RV lead body comprises a passageway extending between a proximal port along the proximal end portion and a distal port along the distal end portion, at least one hemostatic valve located within the passageway, and a removable pusher slideably engaged within the passageway and removably coupled to the LV stimulator to advance it distally from the lead body through tissue.

Another aspect of the present disclosure is a method for assembling a cardiac stimulation system, including coupling a left ventricle stimulator at a location within a left ventricle conduction system to an energy source via a right ventricle (RV) lead assembly extending transmuscularly from an RV chamber to the location.

Additional aspects are contemplated of methods corresponding to the various aspects, modes, embodiments, and features described hereunder with respect to systems and devices presented.

Another aspect of the disclosure is a cardiac stimulation system with a left ventricle (LV) stimulator configured to be positioned transmuscularly from a right ventricle (RV) chamber, and to a location along an LV wall associated with an LV conduction system, and that is configured to be actuated so as to couple energy to cardiac tissue sufficient to stimulate principally the left ventricle from the location.

Another aspect is a cardiac stimulation system with a first right ventricle (RV) stimulator affixed at a first location along a right side of an interventricular septum; a second left ventricle (LV) stimulator affixed at a second location to a left side of the septum; and an elongated member extending across the interventricular septum between the first and second stimulators at the first and second locations, respectively.

Another aspect is a cardiac stimulation system with an RV lead body with a proximal end portion and a distal end portion terminating in a distal tip; a tissue fixation member at a first location along the distal end portion and proximally of the distal tip; and a cardiac tissue stimulator at a second location along the distal end portion. The second location is distally adjacent the first location such that the tissue fixation member when affixed to tissue is adapted to secure the cardiac stimulator at a fixed position within a ventricular wall. The cardiac stimulator is adapted to be coupled to an actuator at a remote location from the fixed position and to be actuated by the actuator in a stimulation mode of operation to stimulate cardiac tissue at the fixed position.

According to one further mode of this aspect, the tissue fixation member comprises a ridged outer surface along the distal end portion. According to one embodiment of this mode, the ridged outer surface is helical along a length and is adapted to be screwed into tissue. In a further embodiment, the lead body comprises an internal passageway with a distal port at a distal tip of the distal end portion, and a hemostatic valve within the passageway at or adjacent to the distal port, and the valve is configured for a guidewire to be positioned therethrough in an open configuration but that is configured to prevent fluid egress into the passageway in a closed configuration.

Another contemplated highly beneficial mode of the various aspects of systems and methods herein presented further provides a transseptal delivery system configured to deliver a stimulator of the system across an interventricular septum at least for placement at a subendocardial location on a left side of the septum. In one embodiment, the transseptal delivery system comprises a transseptal sheath, in another a transseptal needle, and in another both.

According to another mode of the present system aspects, such system is configured for biventricular pacing.

In another mode, the LV stimulator is configured to extend transseptally across the interventricular septum and across the LV to a second location along a free wall of the LV.

In another mode, the LV stimulator comprises a pointed distal tip and distal taper adapted to push through tissue.

In another mode, the LV stimulator comprises a distal taper of distally reducing diameter, and a proximal taper of proximally reducing diameter.

In another mode, the LV stimulator comprises an expandable member that is configured to expand upon breach across the septum into the LV so as to engage an expanded surface area along the LV endocardial wall upon advancement thereagainst. In one embodiment of this mode, the LV stimulator comprises an extendable array of electrodes.

The various aspects, modes, embodiments, variations, and features just described are to be considered independently beneficial without requiring limitation by the others. However, further combinations and sub-combinations between them as may be apparent to one of ordinary skill are also contemplated as further aspects hereunder. Other beneficial aspects, modes, and embodiments are to be appreciated by one of ordinary skill based upon further review of the disclosure below, appended claims, and accompanying Figures.

### BRIEF DESCRIPTION OF THE SEVERAL VIEWS OF THE DRAWING(S)

The invention will be more fully understood by reference to the following drawings which, though variously demonstrating certain highly beneficial embodiments of the present invention considered of independent value, are also provided for illustrative purposes with respect to the broad aspects of the invention and are not intended to necessarily limit those broad aspects except where indicated as such.

FIGS. 1A-C show schematic representations of one single RV lead transseptal biventricular pacing assembly of the present disclosure in each of three respective modes of use in context of a heart.

FIG. 2 shows a schematic representation of another single RV lead transseptal biventricular pacing assembly of the present disclosure, also in context of a heart.

FIG. 3 shows a schematic representation of another single RV lead transseptal biventricular pacing assembly of the present disclosure, according to certain particular features during one mode of use thereof, also in context of a heart.

FIG. 4 shows a schematic representation of another single RV lead transseptal biventricular pacing assembly of the present disclosure, according to certain particular features during one mode of use thereof, also in context of a heart.

FIGS. 5A-B show schematic representations of another single RV lead transseptal biventricular pacing assembly of the present disclosure in each of two respective modes of use in context of a heart.

FIGS. 6A-B show schematic representations of another single RV lead transseptal biventricular pacing assembly of the present disclosure in each of two respective modes of use in context of a heart.

FIG. 7 shows a schematic representation of another single RV lead transseptal biventricular pacing assembly of the present disclosure in each of two respective modes of use in context of a heart.

FIGS. 8A-B show schematic representations of another single RV lead transseptal biventricular pacing assembly of the present disclosure in each of two respective modes of use in context of a heart.

FIGS. 9A-D show schematic representations of another single RV lead transseptal biventricular pacing assembly of the present disclosure in each of four respective modes of use in context of a heart.

FIG. 10A shows a top perspective view of a distal end portion of another single RV lead transseptal biventricular pacing assembly of the present disclosure.

FIG. 10B shows a partially longitudinally cross-sectioned view of the assembly shown in FIG. 10A.

FIG. 10C shows a side view of one component part of the assembly shown in FIGS. 10A-B.

FIG. 10D shows a longitudinally cross-sectioned side view of another component part of the assembly shown in FIGS. 10A-B.

FIG. 10E shows a transversely cross-sectioned view taken through the component part shown in FIG. 10D.

FIG. 10F shows a longitudinally cross-sectioned side view of another component part of the assembly shown in FIGS. 10A-B.

FIG. 10G shows a transversely cross-sectioned view of the component part shown in FIG. 10F.

FIGS. 11A-D show photographs of a physical embodiment constructed according to similar features as the assembly shown in FIGS. 10A-B, during each of four respective modes of use in a bench top demonstration.

FIG. 12 shows a partially longitudinally cross-sectioned side view of a distal end portion of another single RV lead transseptal biventricular pacing assembly of the present disclosure.

FIG. 13A shows a desired site of initiation for LV pacing in a septum of a heart.

FIG. 13B shows another transseptal LV pacing assembly of the present disclosure in one mode of use positioned to initiate pacing of the LV at the location in the heart shown in FIG. 13A.

FIGS. 14A-B show photographs of a transseptal LV pacing assembly in decreasing degree of magnification, respectively, as constructed for use in an experiment conducted according to the present disclosure.

FIGS. 15A-C show photographs of captured fluoroscopic images taken at three sequential modes of operation, respectively, in conducting an experiment in a pre-clinical chronic implant study using an assembly similar to that shown in FIGS. 14A-B.

FIG. 16A shows a photograph of a heart taken post-mortem from a subject following transseptal placement of an LV pacing lead similar to that shown in FIGS. 14A-B as a chronic implant according to a procedure similar to that shown in FIGS. 15A-C, taken with a view from the RV side of the septum through a surgical incision through the free RV wall.

FIG. 16B shows a photograph of the heart shown in FIG. 16A, taken from the LV side view through another surgical incision through the LV free wall, and shows a sub-endocardial placement of the LV pacing electrode on the left side of the septum following a transseptal delivery and placement.

FIG. 16C shows an exploded higher magnified view of the photograph shown in FIG. 16B, showing more detail of the LV pacing electrode.

FIGS. 17-24 show respective graphs of certain data comparisons according to the experiment conducted under Example 1 of the disclosure, comparing various pacing sites alone and in various combinations of multi-site pacing as a function of measurements reflecting cardiac contractility and LV function.

### DETAILED DESCRIPTION OF THE INVENTION

Referring more specifically to the description and drawings provided in the Figures appended herewith, this present disclosure variously provides certain details of various beneficial embodiments illustrative of one or more aspects and modes herein contemplated. While each is considered independently beneficial, additional combinations and sub-combinations between the disclosures and related Figures and illustrations are also contemplated.

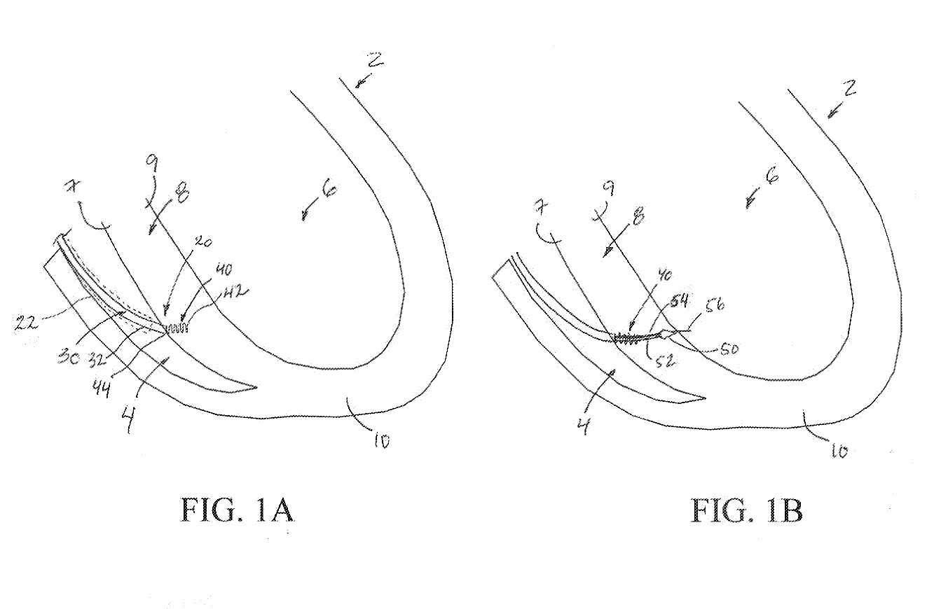

It is to be appreciated that certain illustrations in the Figures (such as for example FIGS. 1A-9D) present various aspects of the present disclosure in context of a heart, and thus reveal certain cardiac features associated with such environment as follows. More specifically, a heart 2 is variously shown to include a right ventricle 4, a left ventricle 6, and a ventricular septum or septal wall 8. The septum 8 is shown to include a right side 7 and a left side 9 associated with the right and left ventricles 4, 6, respectively. These right and left sides 7, 9 of the septum 8 are also illustrated to reflect right and left bundle regions of a purkinje fiber system associated with cardiac conduction. These finer anatomical features (purkinje conduction bundles) are not shown in order to provide clarity of perspective regarding the interventional devices shown in these regions. However, it is to be appreciated by one of ordinary skill that normal conduction propagates into the ventricles via these right and left bundles that are substantially isolated in conducting down along the septum toward the cardiac ventricular apex 10, where they bifurcate along the respective right and left chamber walls for pumping contraction.

These various cardiac features just described are reflected in multiple different Figures, and are given similar numerical reference for purpose of consistency and simplicity in overall understanding among and between the various present embodiments. In addition, where similar general features are provided between different embodiments, they may be given similar numerical reference also for efficiency of understanding. However, it is to be appreciated by one of ordinary skill that despite a feature of one embodiment sharing a similar numerical reference to another generally like feature of another embodiment, finer details of each such feature may vary as appropriate to suit the specific embodiment addressed, respectively; thus, while considered “generally similar” features, they may not necessarily be exactly the same as to such finer details. It is noted that the terms “RV” and “LV” as used hereunder refer to “right ventricle” and “left ventricle” respectively, whereas use of such terms as modifiers to structures named will reference the respective chamber or side of the heart where that structure is associated. For example, “RV electrode” references an electrode used in the RV conduction system, such as for further illustration in a transseptal dual electrode biventricular pacing scheme the RV electrode designates the implanted element on the RV side of the septum.

In addition, the terms “proximal” and “distal” are used throughout this disclosure, and have certain particular contextual meaning depending upon the use as relative terms. With respect to a location relative to a medical device described or physical operation performed by a surgeon or other healthcare provider using such device or operation, “proximal” is intended to mean closer to the user (or further away from the body of the subject or patient), whereas “distal” is intended to mean further away from the user (and thus closer toward the body of the subject or patient). When these terms are used in the context of referencing a physiologic or anatomical feature or process, the term “proximal” generally refers to closer to the body of the patient or “upstream” in the process, whereas the term “distal” generally is intended to mean further away from the patient's body or “downstream” of the process. In particular context for example of the electrical conduction system of the heart, “proximal” in the conduction system means upstream along a conductive wave, whereas “distal” is intended to mean “downstream” along the wave. Further to a conduction “system” as such, actual conductive waves may not be present (e.g. in the setting of block), or may be present in a particular condition that is abnormal and inverted regarding direction than is considered normally physiologic. Unless so addressed specifically to intend a different context to accommodate such abnormal conditions, “proximal” means upstream and “distal” means downstream in context of the normal physiologic conduction condition.

Certain common threads among the embodiments as shown in the Figures are also noted. For example, FIGS. 1A-12 show various embodiments that each provide a biventricular pacing system and method via a single right heart lead system that secures a right heart stimulation electrode on the right side 7 of the ventricular septum 8, and extends a left heart stimulation electrode transmuscularly therefrom to capture the left side 9 for left ventricular stimulation.

More specifically, FIGS. 1A-6B show certain embodiments implanting the left side electrode transmuscularly to the left side 9 of the ventricular septum or septal wall.

Still more specifically, FIGS. 1A-C show one embodiment of the present disclosure in various modes of use within heart 2 as follows.

As shown in FIG. 1A, a single biventricular pacing lead 20 is shown to include an elongated lead body 30 with a distal end portion 32 that includes an RV electrode 40 that is an active fixation electrode. In the particular embodiment shown, which is considered of particular significant benefit, this RV electrode 40 is an electrically conductive helical screw that includes a sharpened distal tip 42 and that is secured to distal end portion 32 of body 30 at proximal junction 44. Lead 20 is deliverable to a site in the right ventricle 4 for implantation of screw 40 on right side 7 of ventricular septum 8 via a delivery sheath 22.

Delivery sheath 22 is shown in shadow as various delivery platforms and techniques apparent to one of ordinary skill may be incorporated together with the various aspects and features of the present embodiments for delivering the electrodes to the desired location. Moreover, various specific features not shown in various embodiments, such as internal lead structures, e.g. conductor wires, torsion modalities, etc., are considered to be included though may not be specifically shown. Such various further approaches and finer features apparent to one of ordinary skill upon review of this disclosure in its entirety are considered within the scope of the present disclosure and not departures from the present disclosure, even if differing from such specific implements shown in order to provide clarity and context to the embodiments in their intended overall systems and uses.

Once RV electrode 40 is taken to the site along right side 7 of septum 8 for desired implantation, it is advanced against the septum while providing torsional turning, to thus screw it into the muscle at that location. A stop may be provided such as for example at proximal junction 44, or adjacent thereto, in order to provide user feedback when complete deployment and active fixation is imbedded within the muscle. Subsequent to such fixation to implant RV electrode 20 along right side 7 of the septum 8, a second LV electrode is then extended through the inner bore (not shown) of coiled screw 20 and transmuscularly across the septum for subsequent implantation at a location adapted to stimulate the left side conduction system. In the present embodiment shown, this is accomplished at a location extended across the septum 8 and at left side 9 for capturing the left side purkinje system in that structure. However, as will be appreciated in further embodiments shown and described hereunder, other transmuscular placements may be accomplished for biventricular pacing success.

Further to the specific mode shown for the present embodiment in FIG. 1B, an LV electrode 50 is advanced from within RV electrode 40 via a pusher 56 that includes a distal tip 56 engaged through a lumen through left side electrode 50 (not shown) but with an enlarged proximal side or stop that confronts electrode 50 to allow an interference with the lumen. This allows for pushing advancement of the LV electrode 50 through cardiac muscle tissue, but slideable withdrawal of pusher 54 to remove it from LV electrode 50 for successful implantation. Distal tip 56 may also be provided with a sensor, e.g. as a simple exposed conductive region of a metal construction for pusher 54 (with proximal shielding for example to isolate the tip 56), which may allow for mapping or “testing” of the location for implantation. This may allow for example adequate feedback when the septum 8 is breached via electrical signals or impedence monitored at the tip 56. It is to be further appreciated with appropriate geometries and length for pusher 54 that, per this sensing mode, it may be extended from the overall lead system 20 prior to active fixation of RV electrode 40 in order to determine the appropriate level along the septum for implantation of the dual electrode, right/left stimulation system (e.g. by testing distal or proximal along the septum to ensure stimulation is distal in the conduction system from any relevant block on either or both sides).

As also shown in FIG. 1B and furthermore in FIG. 1C, an LV conductor lead 52 is coupled to LV electrode 50 and is also extended from within right side electrode 40 as pusher 54 advances LV electrode 50 across the septum 8. As shown in FIG. 1C, once the proper location is achieved for LV electrode 50, pusher 54 is then withdrawn (not shown) to leave the single right heart lead assembly 20 successfully implanted with RV electrode 40 and LV electrode 50 respectively implanted on right and left sides 7,9 of septum 8, and bridged by LV conductor lead 52 extending across the septum 8 between them.

It is to be appreciated that the RV and LV electrodes 40,50 are to be electrically isolated from each other within lead 20, and are together coupled to a pacemaker at isolated lead couplers provided thereon or in. In stimulation mode, the stimulation of each of these electrodes may be in synchrony, or the timing of their pulses may be slightly offset as appropriate for ultimately an objective to achieve physiologically optimal pacing of the respective ventricles in a particular patient.

While various existing pacemakers are programmable to accommodate particular patient needs or adjunctive device interface such as pacing lead(s), it is contemplated that the dual electrode/dual pacing lead of the present embodiment (and of other embodiments elsewhere herein shown and described) may be suitably combined with such existing pacemakers. Or, a custom pacemaker may be appropriately developed by one of ordinary skill for specified combined use with a particularly executed physical lead system consistent with the various broad aspects, modes, or embodiments of the present disclosure. In either regard, however, it is contemplated that the lead may be provided in combination with, or packaged separately for later combined use with, one or more such pacemakers. In such cases, instructions for use may be provided that describe various functional aspects of the lead such as impedance of the respective electrodes in order to optimize appropriate combined use with such pacemaker(s).

It is noted further to the embodiment of FIGS. 1A-C that LV electrode 50 is shown to have a diamond geometry. While this particular approach is considered useful, it is also schematically representative of certain features considered to provide certain benefits more broadly. For example, a pointed distal tip is thus provided, which allows for piercing through muscle of the septum 8 during advancement with pusher 54. Furthermore, a proximal taper is also provided. This feature may be adapted in a manner that enhances extraction of the electrode 50, either in order to redeploy due to inappropriate positioning determined through testing or pacing results, or for other reasons that may occur to require removal of a lead altogether from a patient (and which may be some extended period after the implantation procedure and some chronic function). Under whatever circumstance giving rise to the need, however, the proximal taper allows similarly enhanced proximal movement through the septum toward the right side 7, such as may be achieved for example by pulling on the LV conductor lead 52. Moreover, it is further noted as to the “removability” benefit, that such conductor 52 and its coupling to LV electrode 50 be of such sufficient construction so as to allow removal of LV electrode 50 through tissue upon applying tension to conductor 52.

As noted above, the dual electrode/dual pacing, single lead system 20 just described provides a highly beneficial right side active fixation RV electrode 40 which as a hollow screw allows for internal housing of an extendable LV electrode to be advanced across the septum to capture the left side conduction system for transseptal biventricular pacing. However, the specific electrode structure used for such left side conduction may be varied. Certain particularly beneficial further embodiments for this feature are contemplated and presented hereunder for further illustration.

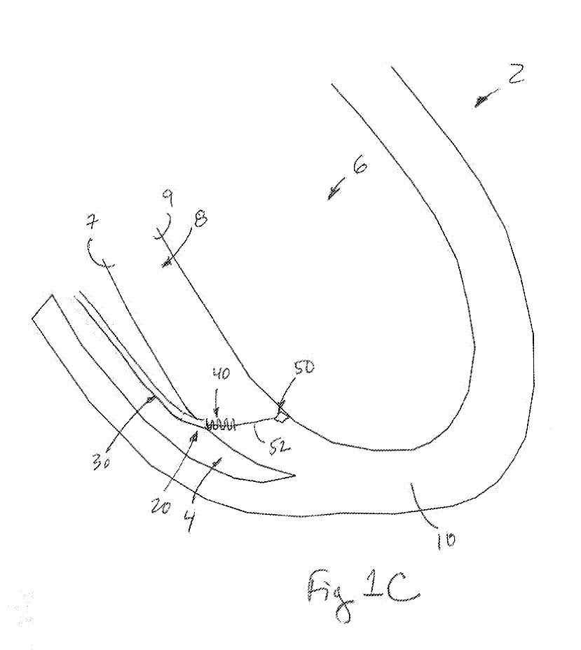

One particular such further embodiment is shown in FIG. 2. Here, various features of right heart lead system 20 are similar to the prior embodiment of FIGS. 1A-C. However, in this present embodiment, LV electrode 60 is a second helical screw geometry. While the specific mode shown in FIG. 2 is “as deployed” across the septum 8, it is appreciated that, prior to deployment, this screw-type LV electrode 60 may housed within the bore of RV electrode 40 and advanced across the septum 8 via a twisting action screwing it in and through the tissue. In this regard, while not necessarily required, it is contemplated for example that a torsion tool (not shown) may be deployed to assist in providing sufficiently robust stiffness of construction and forces for this journey across the structure, but to be removable in order to provide a more flexible extension across the septum as an implant.

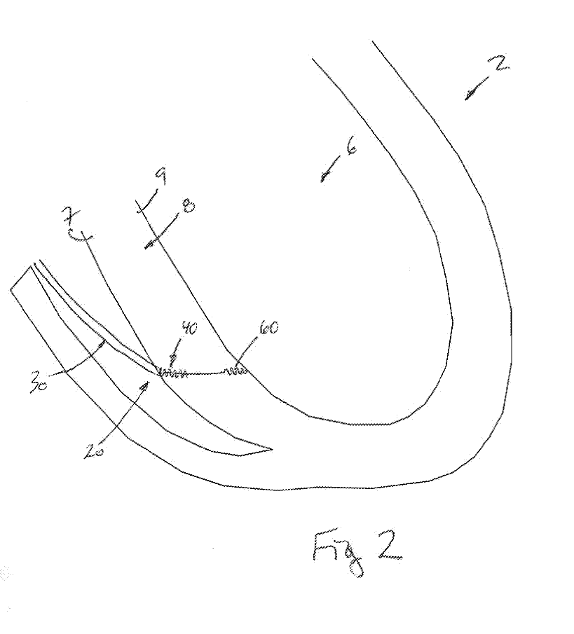

In still a further embodiment shown in FIG. 3, LV electrode 70 is provided as an array of electroded, extendable splines, such as schematically shown at splines 72, 74, 76. In the particular illustrative embodiment shown, these splines extend distally through the inner bore of screw-type RV electrode 40 and across septum 8 in a diverging curved geometry that allow them to span across a region along the right side 9 of septum 8. The ends or location(s) along these splines may be electroded, such as by exposing an electrically conductive metal of the construction that is elsewhere shielded such as by insulation or cladding. Various specific constructions may be implemented by one of ordinary skill to achieve a suitable result consistent with the overall objectives of the present embodiment. However, for further illustration, one particularly beneficial approach contemplated includes providing the splines of the array as pre-shaped superelastic nickel titanium alloy hypotubes with highly conductive metal extending through the hypotube bores either through tips where they are exposed or otherwise exposed through window(s) through the hypotube to provide the stimulation electrode. In one more specific example for further illustration, such hypotubes may be swaged onto an inner conductor that is extended therefrom in a sharpened tip. In another more specific example, such shaped hypotubes may provide a guide for placing the leads, after which the hypotubes are removed with the leads left in place. Is still another alternative approach, the splines of the array may be of a more flexible type of material construction, and guided through the tissue in the desired geometry via shaped internal mandrels.

It is also contemplated that an array of multiple such extended electrodes need not necessarily be provided. For example, an electrode and delivery modality such as just described above for arrayed splines may be employed but as a single stimulation electrode for the left side stimulation. Moreover, such may be straight, or may be shaped as described for the array embodiment. In this latter regard, this allows for example the benefit of active fixation of the RV electrode 40, with some freedom to place the LV electrode (of whatever specific embodiment employed) in a manner that is “off-axis” from the right sided fixation of the lead 20. Still further, by providing sensing and signal monitoring in such shaped extended left side delivery modality, a freedom of customizing (or adjusting) such deployment may be recognized after fixation of the right side.

It is appreciated that, in any specific mode of the present embodiments, providing an extendable LV electrode through the internal bore of an outer RV electrode that is an active fixation electrode such as a screw provides certain specific benefits not previously provided by other systems. In one particular regard, by providing the right side at the larger radius, it provides a more robust fixation and construction to anchor the assembly of the overall lead system 20 during manipulation of the left sided placement. Furthermore, this right sided fixation is the primary point of engagement between the lead body 30 and the septum 8 where the dual electrodes are implanted. In a beating heart environment over extended life and chronic wear, this engagement and transition between septum to the lead body must be of robust construction and withstand chronic fatigue without failure or substantial degradation. However, the LV electrodes of these particular embodiments extend within an imbedded muscle structure of the septum. Accordingly, it is believed that providing an outer screw electrode for right side fixation, with an extended left side electrode from within the screw, is considered to provide certain particular benefits over an alternative arrangement for example which might instead provide an internal screw for right side fixation and stimulation that is located within a wider radiused exterior array of extendable electrodes for transmuscular left side deployment.

While not shown in the overall assembly illustrated in FIG. 3, the LV electrode array 70 of angled or curved splines may be held in a confined and relatively straight geometry prior to extended deployment within an outer sheath located around the array but within the coil of RV electrode 40. Alternatively, the helical wall of RV electrode 40 may be provided with sufficient construction and geometry to provide such confinement prior to extended deployment and superelastic recovery to their curved shape in the muscle tissue of septum 8. In addition, other uses of such internal delivery sheaths for the LV electrode embodiments are also contemplated.

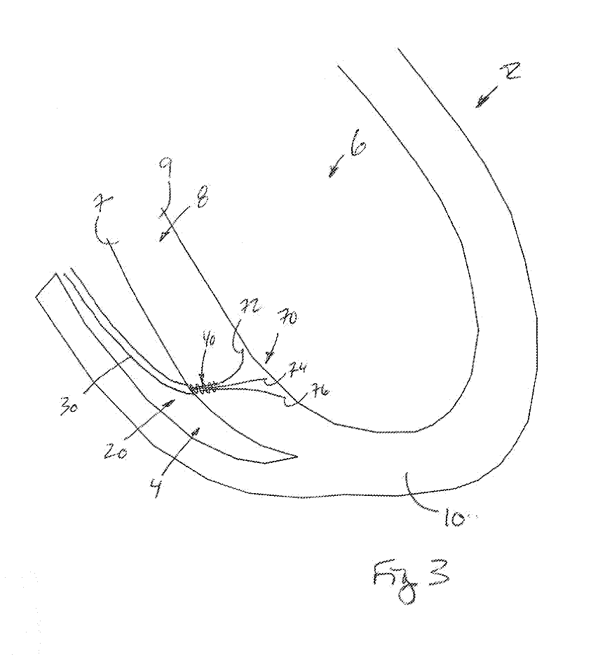

For example, FIG. 4 shows a dual electrode/biventricular pacing, single right sided lead system 20 that includes an actively fixated RV electrode 40 as a helical screw, but with an internal sheath 80 extended therefrom and across the septum 8 to the left side 9. Sheath 80 includes a sharpened tip 82 to aid in such advancing deployment. Sheath 80 also includes a lumen 84 in which a left side electrode may be housed. Accordingly, by removing the sheath 80 from the position shown in FIG. 4, but without removing the internal electrode delivered to that location with the sheath, the electrode is left in place.

Furthermore, such a sheath 80 may also provide complete transmuscular delivery across and through the septum 8 and into left ventricle 6. In doing so, certain electrode assemblies may be so deployed for subsequent fixation to the left side system via the endocardial surface of the left ventricle 6. This is shown for example in various further embodiments as follows.

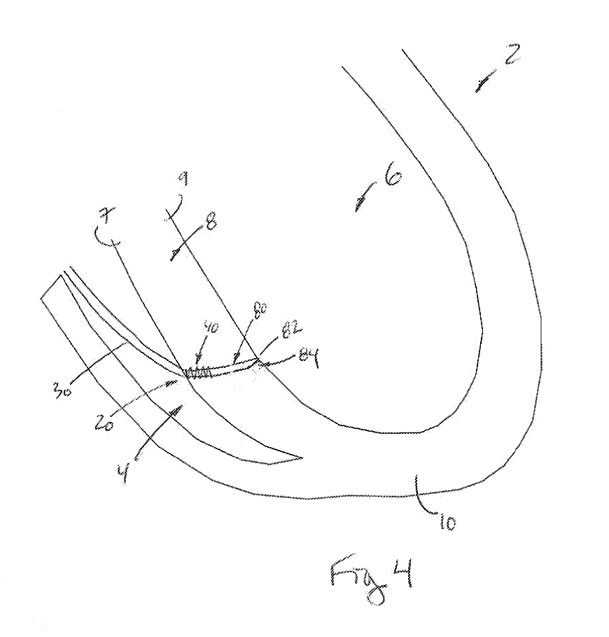

FIG. 5A shows a dual electrode/biventricular pacing, single RV lead system 20 that includes an RV electrode 40 similar to that previously described above, but including an LV electrode 90 that is extended completely across septum 8 and with a curved or hooked tip 92 located in left ventricle 6. By providing such interim deployment, the LV assembly 90 may be thereafter withdrawn proximally toward the septum 8 to imbed the hooked tip 92 into the endocardial wall of the septum 8 on its left side 9, as shown in FIG. 5B. The tip 92 is provided with an isolated electrode to provide stimulation, which may be either only on the tip portion distal to the curved portion, or include all or a part of the curved portion of the hook, and/or may include a proximal portion adjacent the curve, or may extend along a length that includes all of the above. Regardless of the specific location of the electroded segment, it is contemplated nonetheless that this arrangement provides for an overall robust fixation of the electrode on the left side, and ensures a well captured left side via the endocardial left sided engagement. Moreover, subsequent advancement of a needle-type delivery sheath such as shown in FIG. 4 may be employed to push the hook back out from the left side 9 of the septum 8, followed by retraction back into the needle sheath, and subsequent removal if required for any reason.

Other specific approaches similar to that just described, but with further variations and left side electrode assemblies, are also contemplated.

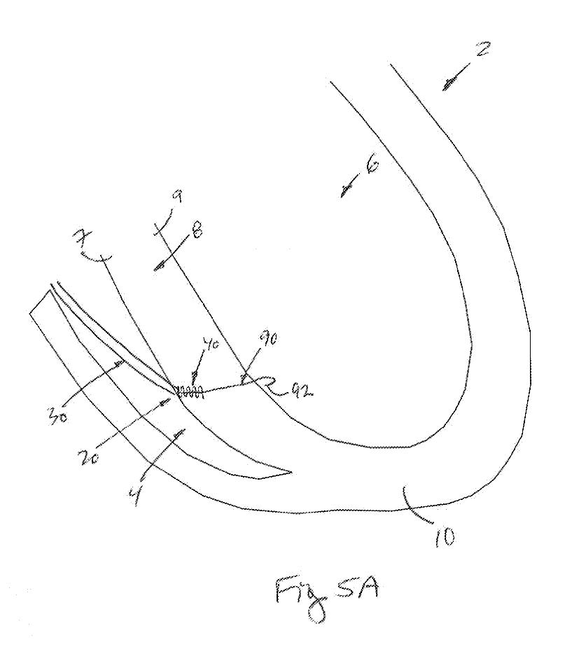

For example, FIG. 6A shows a similar arrangement as shown in FIG. 5A, except involving a different LV electrode 100. In this particular embodiment, the LV electrode 100 shown in the left ventricle chamber 6 includes an expanded array of proximally oriented splines 102. Further to this specific illustrative embodiment, these “rearward” facing splines 102 are coupled to a fabric or membrane 110. By withdrawing this assembly proximally in the chamber of left ventricle 6 toward septum 8, the array is embedded into the endocardial surface of left side 9 of septum 8, as shown in FIG. 6B. The specific electrodes along the splines 102 may be at their tips, or elsewhere, so long as robust left sided capture and stimulation may be achieved. In fact, by providing the membrane 110 on an exterior of the splines 102 relative to chamber 6 of left ventricle, the stimulation electrode portion may be seated on the actual endocardial surface of the left side 9, but be beneficially insulated by the membrane from blood in the ventricle during electrical stimulation or shocking. In another regard, by embedding the splines within such a membrane or fabric, other than where engaged into the septal wall tissue, corrosion and wear resistance may be enhanced. In any case, in addition to the various benefits presented by this current embodiment, certain additional benefits also provided by the electroded array arrangement spanning an area of the left side 9 for stimulation, such as previously presented for the FIG. 3 embodiment, are also implicated in this further embodiment.

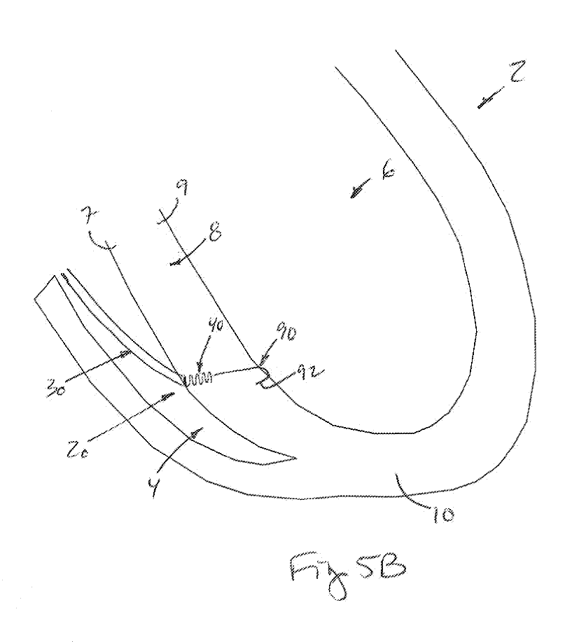

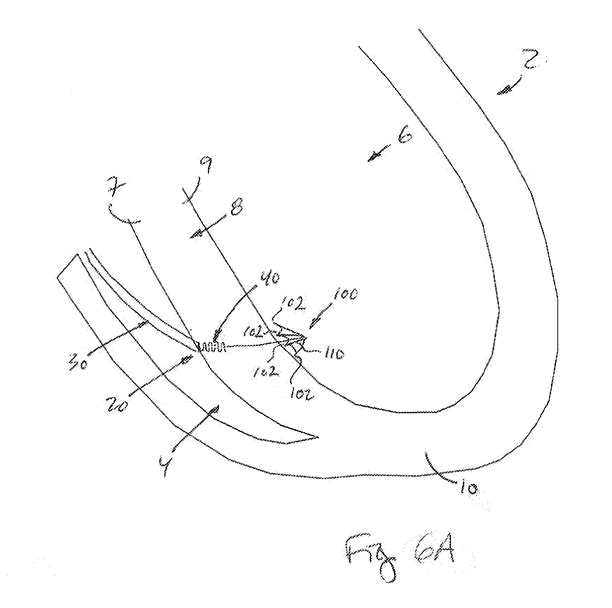

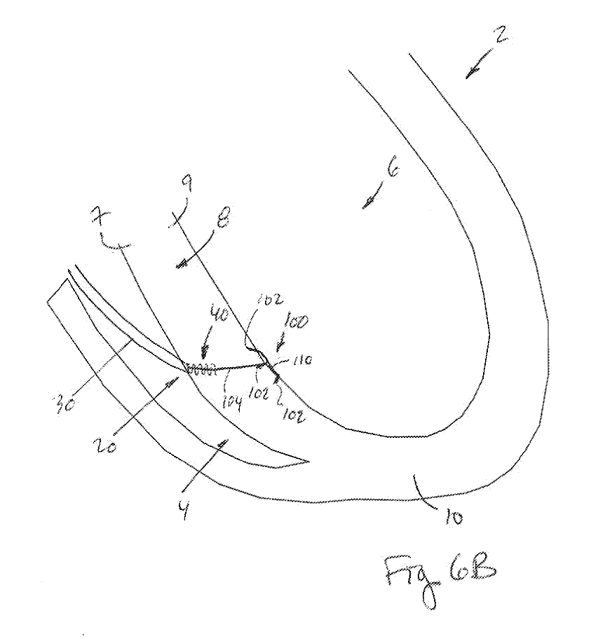

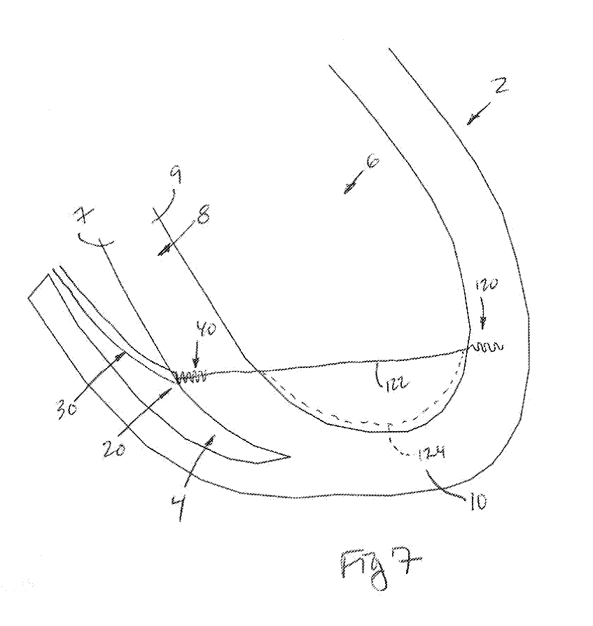

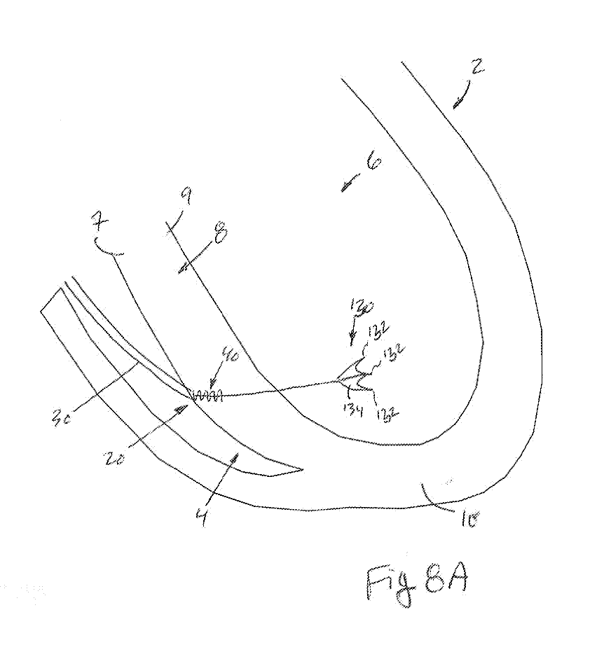

In contrast to the modes shown for the previous embodiments of FIGS. 1A-6B providing septal pacing for both right and left sides of the heart via a single right sided lead, FIGS. 7-8B show various embodiments extending the LV stimulation electrode from an actively fixed right side electrode, through the septum for implantation across the left ventricle 6 in the far wall of that chamber.

More specifically, FIG. 7 shows a similar dual stimulation electrode, single right sided lead system 20 as previously described for the various preceding embodiments above, but with an LV electrode 120 extended across ventricle 6 where it is actively fixated to the far wall of ventricle 6 opposite septum 8. in this arrangement shown, conductor lead 122 thus extends across the septum 8 and chamber of left ventricle 6 between the RV and LV electrodes 40,120. According to this chronic exposure to the blood in the left ventricle 6, the exposed surface of this LV conductor lead 122 may be beneficially treated for particularly robust biocompatibility and thromboresistance, such as via a coating or endothelialization promoting surface (such as for example surface antibodies for endothelial precursor cell (EPC) recruitment or binding/adhesion, such as are being developed and promoted in clinical use by Orbus Niche for certain stent implants for example). Moreover, a particular shape may be given to this region of exposed lead in the left ventricle 6, such as shown in FIG. 7 at shadowed lead 124 biased against the wall, in order to minimize or at least reduce potential compromise to hemodynamics in the chamber.

In this particular arrangement between RV and LV electrodes 40, 120 shown in FIG. 7, it is to be appreciated that the RV electrode 40 of the system 20 stimulates the right ventricle conduction system along the right side 7 of septum 8, whereas the LV electrode 120 is instead located much more distal in the left side conduction system well past the septum 8 and apex 10. In this regard, the pumping action of left ventricle 6 may be sub-optimal versus ideal pumping contraction that is initiated in a more physiologic manner at the left side 9 of septum 8. However, in many cases disease in the conduction system giving rise to the need for stimulation in the first place may prevent successful pacing or defibrillation via the septum 8 as it is more proximal than disease or block in that conduction pipeline. Accordingly, this present arrangement may be of particular benefit in such circumstances. Moreover, while more distal in the left side conduction system than septal pacing, the position of the left side electrode 120 may nonetheless be more proximal, and with better pumping efficiency, than in traditional coronary sinus transvascular approaches. Still further, even in the event the left side electrode were stimulating at a similar location as may be accomplished with such a traditional coronary sinus placement, the present ability to do so via a single right sided lead nonetheless still provides certain distinct benefits.

As with other prior embodiments presented previously above, the specific left side electrode presented by the trans-septal, trans-LV embodiment of FIG. 7 may be modified to include other specific electrode assemblies for the left sided stimulation at such locations. One particular example is shown in FIGS. 8A-B. More specifically, this present embodiment includes a left side electrode assembly 130 that includes an array of electroded splines 132 that are covered by a covering or membrane 134. This is a similar arrangement as that described previously above by reference to FIGS. 6A-B, except providing the “tented” array in a distal orientation. This allows distal advancement of the assembly of this present embodiment across the left ventricle 6 from the septum 8 to imbed the splines 132 into the far left ventricle wall, as shown in FIG. 8B. As with other transseptal/trans-LV delivery per other embodiments hereunder, the initial delivery across the septum from the right ventricle 4 may be accomplished for example via an extendable needle such as shown at needle 80 in FIG. 4 (and which confines the spline array to a collapsed configuration prior to self expansion when released through the septum 8 into left ventricle 6). Various of the finer features and benefits presented previously with respect to FIGS. 6A-B thus apply similarly with respect to this present embodiment, with the added aspects related to the trans-LV delivery and different placement for left heart stimulation and contractile conduction.

While the prior embodiments above introduce biventricular pacing of both right and left sides from the septal wall, and with the left side extended across the left ventricle to more distal conduction locations on the far left ventricle wall, still further other arrangements are also contemplated.

More specifically, according to still further embodiments of the present disclosure, FIGS. 9A-D show a more distal orientation and location for the right side electrode and providing for extended transmuscular implantation of the left electrode to capture the left side conduction system around the area of the ventricular apex 10 as follows.

As shown in FIG. 9A, a similar dual electrode, single right sided lead system 20 as prior embodiments is presented with a body 30 terminating distally in a helically coiled screw tip RV electrode 40. However, in the present embodiment, this assembly is delivered via a delivery sheath 24 that has its distal tip 26 engaged distally in the right ventricle 4 around the apex 10. As shown in FIG. 9B, RV electrode 40 is thus delivered to this location and out tip 26 where it is screwed into the muscle tissue there. This represents a location to stimulate the right side conduction system more distally than in prior embodiments noted above, with similar considerations to placement of right side stimulation for particular patients as presented previously with respect to left side placement (e.g., re: location of disease or block within respective conduction system, ability to capture all of the respective conduction system at the location, etc.).

As shown in a subsequent mode of deployment and use in FIG. 9C, a left side electrode assembly 150 is extended from the RV electrode 40 and across the muscle at the apex 10 to a location intended to appropriately stimulate the left side conduction system. In the particular embodiment shown, the electrode 152 and conductor 154 may be generally similar for illustration purposes to those features shown in FIG. 1B-C, provided however that a different pusher mechanism is shown via pusher 156 that is instead an outer sheath around conductor lead 154. Accordingly, it is to be appreciated that many such variations may be made, including those shown in addition to others apparent to one of ordinary skill though not specifically herein shown or described, and still remain within the broad intended scope of the present aspects of the disclosure. In this regard, it is to be appreciated that various of the present embodiments for extendable electrode assemblies for right and left side pacing via a single right sided lead may be used in the trans-apical approach presented by way of illustration via the particular embodiment shown in FIGS. 9A-D. In any case, as the respective delivery mechanism is successfully employed, an extended arrangement between right and left side stimulation electrodes 40,152 such as shown by way of example in FIG. 9D is achieved. This particular approach regarding electrode positioning may be of benefit for example where both right and left bundle blocks (or substantial disease) exists in the septum but where stimulation at or around the apex is sufficient to provide a robust contractile conduction result on both sides.

The prior embodiments above have been presented with particular emphasis on certain configurations, arrangements, and uses of a dual electrode, single right sided lead system 20 is it is employed in a heart 2 for biventricular pacing. Though not shown in each such Figure related to such embodiments, it is appreciated that such lead system 20 includes certain proximal components that couple to a cardiac stimulator, such as a pacemaker and/or defibrillator. For purpose of illustration, such features and couplings are schematically shown in FIG. 9D. While presented in context of this present embodiment, it is to be appreciated by one of ordinary skill that such representation and disclosure apply also with respect to the other embodiments as if bodily included in those related Figures.

More specifically, body 30 of right sided lead assembly 20 includes a proximal end portion 31 that includes an RV proximal lead 32 that bifurcates from an LV proximal lead 35. According to the extendable arrangement between RV and LV electrodes contemplated in the embodiments, these RV and LV proximal leads are adjustable relative to each other, such that the LV proximal lead 35 may be manually advanced relative to the RV proximal lead 32 by an implanting surgeon. In the particular embodiment shown, this arrangement is accomplished via a side port 34 located within RV proximal lead 32 and through which LV proximal lead 35 is slideably engaged into a lumen that traverses body 30 to the inner bore of RV electrode 40. In this fashion, the left side components traverse the lumen such that manual manipulation outside the heart allows advancement of the LV electrode from the RV electrode 40. These RV and LV proximal leads 32, 35 include RV and LV proximal couplers 33, 37, respectively. Such RV and LV proximal couplers 33, 37 are electrically coupled to respective RV and LV couplers 152, 154 of a cardiac stimulator 150, which as noted my be for example a pacemaker or defibrillator capable of biventricular stimulation modes of operation. It is to be appreciated that the couplers may be of industry standard type as may be apparent to one of ordinary skill, or may be customized as appropriate to one of ordinary skill adapting them to a specific implementation consistent with one or more aspects contemplated hereunder (for example, an electroded array embodiment to be treated as “one” side electrode may require custom coupler and/or stimulator features to accommodate appropriately energizing the respective sub-feature electrodes of the coordinated assembly).

Each of the prior embodiments may be constructed according to various specific dimensions, materials, or other finer details regarding their respective component parts in order to carry out the intended structures and related methods described, and such aspects may be appropriately modified to suit a particular implementation, as would be apparent one of ordinary skill.

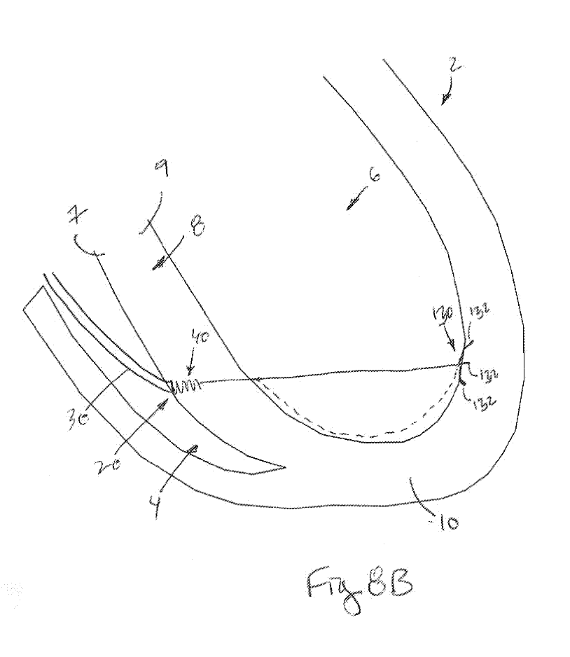

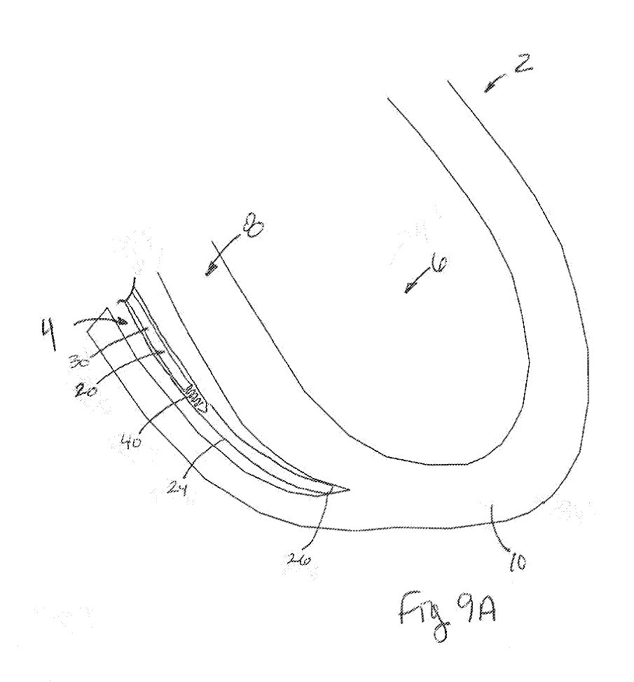

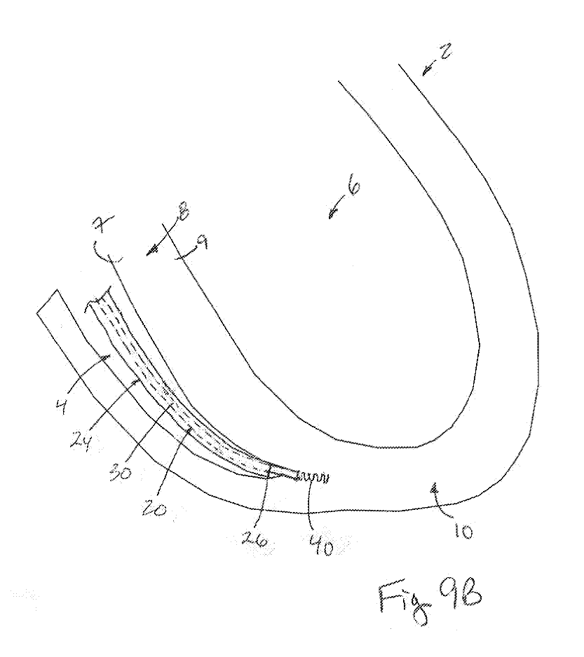

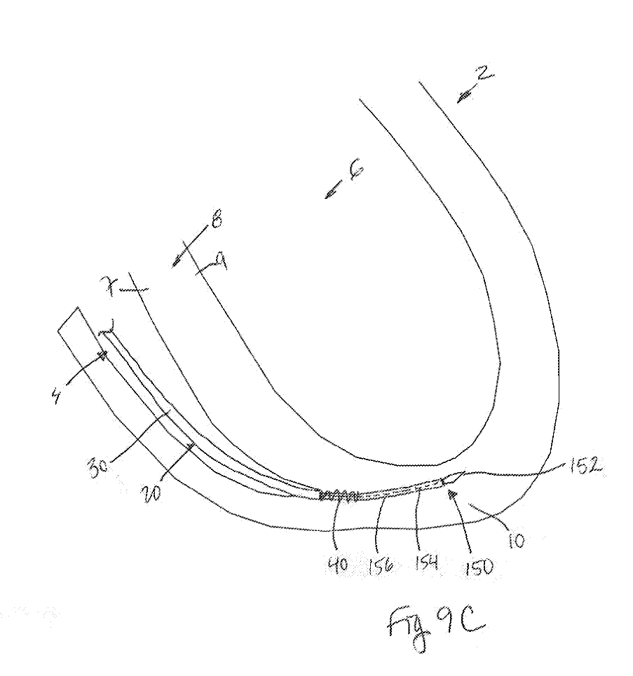

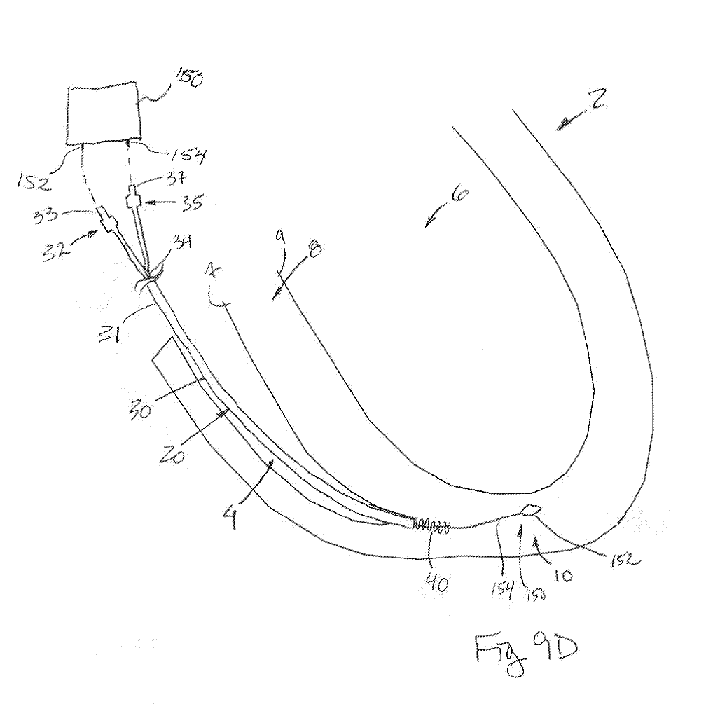

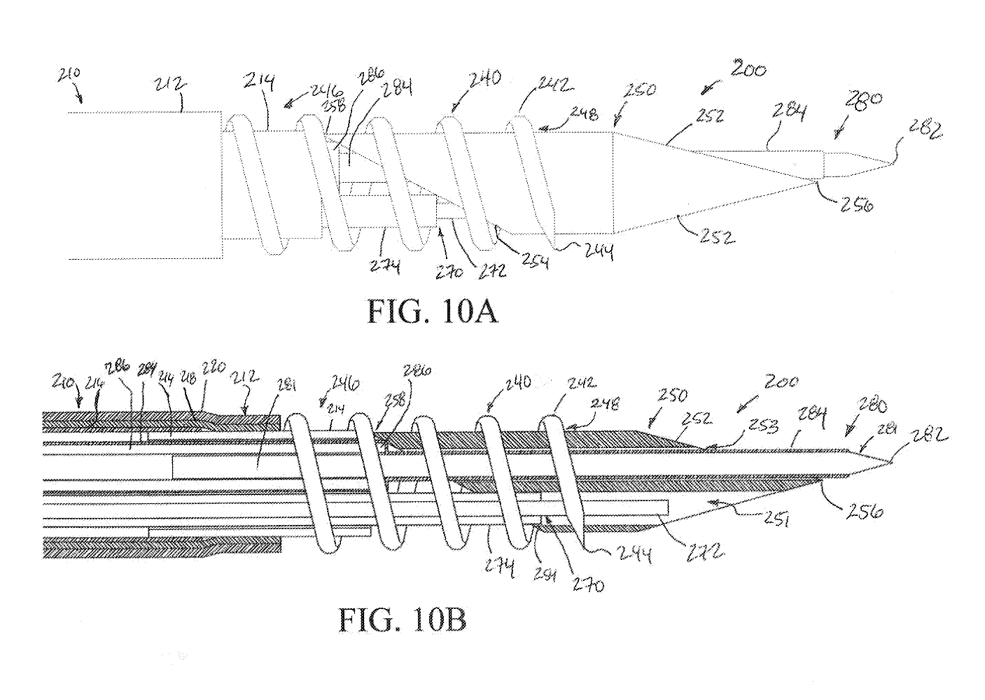

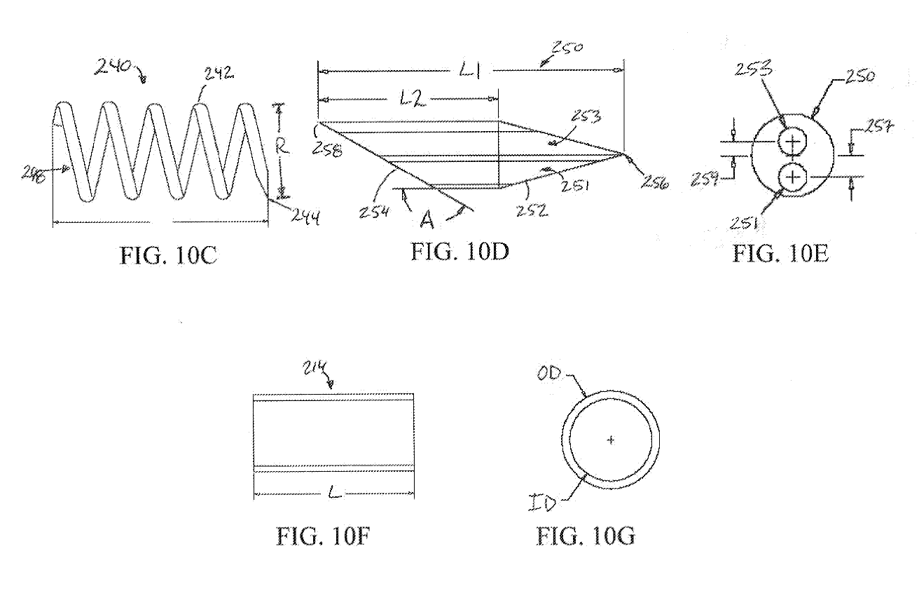

However, for purpose of further illustration, certain finer details of one specific dual stimulation electrode, single right heart lead system 200 is shown in various views in FIGS. 10A-G as follows.

FIG. 10A (and referring to FIG. 10B for certain finer detail noted) shows a dual stimulation electrode, single right heart lead system 200 that is similar in certain regards to the embodiment shown in various modes of use in FIGS. 1A-C as follows. Lead system 200 includes a body 210 with a distal end portion 212 that is secured to an RV electrode 240. As shown in finer detail in FIG. 10C, RV electrode 240 includes a helical filament 242 in a coiled spring or screw type geometry, with a sharpened distal tip 244 to enhance distal advancement into and through septal wall muscle as the filament 242 is rotated under an applied distally oriented force. Opposite distal tip 244, RV electrode 240 is secured to the distal end portion 212 of body 210 at proximal junction 246. Extending within helical filament 242 is an inner bore 248.

In general, certain objectives of the RV electrode 240, and which may impact a specific construction implemented, include without limitation: a robust ability to screw into the septal wall tissue and/or remain secured there; suitable corrosion and wear resistance to withstand fatigue failures as a chronic implant in the right interventricular septal wall; and suitable capture efficiency to stimulate the cardiac tissue at suitable impedance levels to operate in this capacity with appropriate excitation impulses. As for the latter objective, the electrode ideally would not present issues in interfacing with the excitation source in their combined intended use (including without limitation and in one significant regard charge depletion over time and battery life of the source pacemaker or defibrillator).

The specific pitch, spacing between windings, size and material construction of the conductive filament forming the screw, and overall dimensions such as length L, and radial diameter D of the particular coil used for screw-type RV electrode 240 may vary to suit a particular implementation and to the extent accomplishing for example one or more of the objectives for the component noted hereunder. The various combinations of these features may also vary, whereas it is to be appreciated that one variable aspect of the coiled screw construction may impact the appropriate specifications for another. For example, an electrode at one length may be most appropriate at a particular radial diameter but at another length may be more appropriately constructed at another diameter, such as for example to achieve a similar impedance or capture threshold in tissue, or appropriate fixation integrity at the septal wall.

Notwithstanding the foregoing, in the particular embodiment shown, the length L of the coiled screw filament of RV electrode 240 is about 0.143 inches long, and the radial diameter R is about 0.063 inches across (though again each or both of these may vary, as may the respective ratio between them). While various conductor materials may be employed, certain physical embodiments have been previously made from 300 grade stainless steel (though as stated other suitable conductors for chronically implanted screw electrodes are contemplated as apparent to one of ordinary skill).

As also shown in FIGS. 10A-B, and in finer detail in FIGS. 10D-E, an LV electrode 250 is located within inner bore 248 of RV electrode 240 and is held there in a friction fit. LV electrode 250 includes both distal and proximal tapers 252, 254, with a sharpened distal tip 256, and a sharpened proximal tip 258. Left side electrode 250 also includes first and second inner bores, passageways, or lumens 251, 253 (shown in the longitudinally cross-sectioned view of LV electrode 250 in FIG. 10D) that extend longitudinally between distal and proximal respective openings in the structure along the distal and proximal tapers 252, 254.

Various specific dimensions and material constructions may be employed for LV electrode 250 and remain consistently within the intended scope of the present embodiment. However, in one particular example for illustration (and by reference to FIGS. 10D-E): length L1 between distal tip 256 and proximal tip 258 may be for example about 0.213 inches, while length L2 shown may be for example about 0.125 inches; angle A of proximal taper 254 may be for example about 150 degrees; overall outer diameter for LV electrode 250 may be for example about 0.047 inches; inner diameters for inner lumens 251, 253 may be for example about 0.016 inches; offset 257 between the center of inner lumen 251 and the center line of LV electrode 250 may be about 0.013 inches; and offset 259 between the center of inner lumen 253 and the center line of LV electrode 250 may be about 0.006 inches (e.g. the lumen 253 border runs tangentially along the central axis to the pointed distal tip 256).

Extending proximally from LV electrode 250 is an LV conductor lead 270, which includes a distal conductive tip section 272 that is secured to LV electrode 250 within its inner lumen 251. LV conductor lead 270 extends slideably within the body 210 along a proximal end portion of the conductor lead 270 that includes an outer electrical insulator 274.

A pusher 280 includes an electrical conductor core wire 281 with an exposed, electrically conductive pointed tip 282 extending beyond an outer electrical insulator 284. Core wire 281 is secured within an outer tube 286 (such as a metal hypotube or other pushable outer tubing member) which provides a stepped up outer diameter to the pusher 280 and that is larger at this outer tube 286 than the inner diameter of the inner lumen 253. Accordingly, pusher 280 is slideably engaged within inner lumen 253 of LV electrode 250 to the extent it is distally advanceable through that lumen 253 and beyond distal tip 256 of LV electrode 250 only up until the outer tube 286 confronts the proximal taper 254 of the LV electrode 250 at the proximal port into lumen 253.

Such advancement of pusher 280 distally through LV electrode 250 may be performed for example into the interventricular septal wall at a potentially desired location for pacing via the RV chamber in order to use exposed conductor tip 282 to map electrical signals at such location to test its location (e.g. distal vs. proximal) relative to a block in the respective conduction system. However, upon confronting engagement of outer tube 286 against LV electrode 250, further advancement of pusher 280 transmits the distal force to LV electrode 250. This allows distal forces and advancement of pusher 280 to advance LV electrode 250 distally from the inner bore 248 of RV electrode 240 to extend therefrom through muscle tissue for a desired transmuscular implantation. Moreover, with the exposed conductive tip 282 remaining extended beyond distal tip 256 of LV electrode 250, further sensing via that tip may be employed to monitor a breach of the septum 8 into the left ventricle 6, thus providing useful feedback regarding relative positioning of the components by reference to a desired endocardial placement of the LV electrode 250 along left side 9 of the septum 8.

The various components just described above are coupled to body 210 of lead system 200, according to various additional components of that portion of the assembly that are provided as follows.

Body 210 further includes an electrically conductive metal hypotube 214 that is located along distal end portion 212 and extends within a proximal portion of bore 248 of RV electrode such that filament 242 is secured onto hypotube 214 to form proximal junction 246 with body 210. Coupled to an outer surface of hypotube 214 within distal end portion 212 and extending proximally along body 210 therefrom is a layered series of (in this order going outward) electrical insulator 216, RV electrical conductor lead 218, and outer covering 220 which may be for example a jacket, tubing, or coating. These outer layers essentially form body 210, with RV conductor lead 218 insulated from inner coupling with fluids or other electrical conductors (e.g. LV conductor lead 270), and from outer coupling with external environs around body 210, by inner insulator 216 and outer covering 220 respectively.

As with other components shown and described, various material constructions and dimensions may be provided for the body components just described according to one of ordinary skill. However, for further illustration, a couple exemplary dimensions are provided as follows. As shown in FIGS. 10E-G, hypotube 214 may include for example a length L of about 0.105 inches, and outer and inner diameters (OD and ID) of about 0.050 and about 0.042 inches, respectively.

While certain proximal aspects of lead system 200 are not shown in these FIGS. 10A-G, they are to be readily accomplished by one of ordinary skill and by reference to the general teachings provided hereunder. In one regard, along a proximal end portion of body 210 RV conductor lead 218 is electrically coupled to a proximal RV electrical connector or coupler, which in turn is coupled by an implanting surgeon to an RV lead coupler of an energy source such as a pacemaker or defibrillator. Similarly, LV conductor lead 270 is proximally coupled to a proximal LV connector or coupler, which in turn is coupled by the implanting surgeon to an LV lead coupler of the energy source. In addition, the slideable relationships of pusher 280 and LV electrode 250 (and associated LV conductor lead 270) relative to RV electrode 240 and body 210 may be accomplished in a number of approaches.

In one particular example, body 210 terminates proximally in a proximal RV coupler that includes an interior port into a through lumen of body 210 through which pusher 280 is slideably engaged. In order to accomplish slideable relative movement with the LV conductor lead 270, the body 210 includes a side port located distally adjacent to the proximal RV coupler and through which the proximal insulated portion 274 of LV conductor lead 270 slideably extends to terminate proximally in the proximal LV coupler separately. According to this overall arrangement, upon advancing pusher 280 through body 210 and distally confronting and advancing LV electrode 250 across a septum 8, the LV conductor lead 270 is able to also advance forward relative to body 210 via its slideable engagement through the proximal side port. This allows such movement of the LV components with the body 210 and RV electrode 240 secured thereto in a stable, fixed position. Following LV electrode deployment, the pusher is then retracted from the assembly and the respective proximal RV and LV couplers are engaged to the excitation source. In addition, at this time, the distal assembly of the lead 200 becomes much more flexible upon removal of the pusher 280 and representing a more robust configuration for the chronic implant in the beating heart.

Other considerations may also be given to a final constructed assembly which, though not shown in detail in the Figures, are considered hereunder and remain consistent with the current embodiments and within the intended scope of the various broad aspects disclosed. In one highly beneficial further feature as such, certain aspects of the overall lead assembly 200 may include valves or seals in order to ensure robust isolation of electrical components, and additionally preventingress of body fluids that may cause corrosion of various components or other unwanted and potentially harmful results in these chronic cardiac implants. In one particular regard, the distal end portion 212 of lead assembly 200 may include one or more internal valves associated with the slideable engagement of each of LV conductor lead 270 and pusher 280 with body 210. In general, such valving allows for slideable engagement through the tip end of the body 210, but prevent fluid ingress at their respective slideable interfaces with the body 210. And, in the case of pusher 280 to the extent it is removed after LV electrode placement and during chronic implant survival, the respective valving preferably shuts down the luminal access enjoyed by the pusher 280 once it is removed. Such valving may be accomplished by many different structures or mechanisms according to one of ordinary skill, but may include for illustration a grommet valve, duck-bill type of valve, or other form of hemostasis valve.

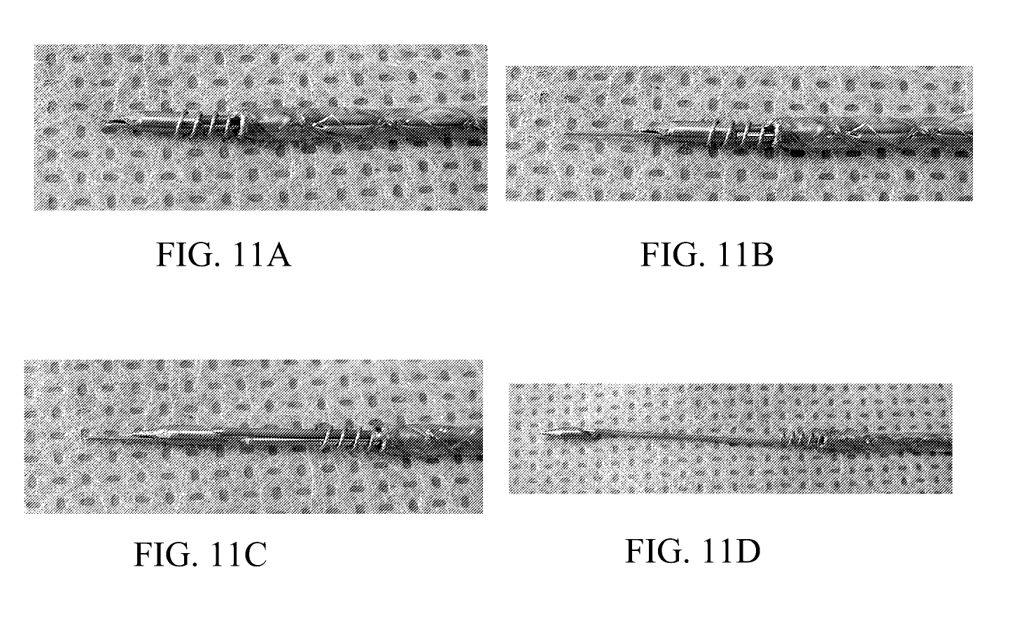

While the previously presented Figures described above provide a robust understanding of certain detailed embodiments to illustrate certain aspects of the present disclosure, for further illustration a physical embodiment of a biventricular pacing, single RV lead system similar to that just described by reference to FIGS. 10A-G above is shown photographically in FIGS. 11A-D during various modes of use according to a benchtop demonstration performed as follows (references to component parts in describing the photos in FIGS. 11A-D refer to similar components in FIGS. 10A-G where comparisons may be readily made visually between the Figures).

More specifically, FIG. 11A shows a distal end portion of an RV lead assembly in a delivery mode similar to that shown in FIGS. 10A-B. FIG. 11B shows the assembly with a distal pointed tip of a pusher extended distally from the respective LV electrode while the LV electrode remains engaged within RV electrode screw that is fixed to the distal end of the lead body. This represents a “probing” mode allowing for mapping and testing of conductivity to test placement for the stimulation electrode on either or both of the right and left sides of a septum. FIG. 11C shows the pusher further extended from the lead body and RV electrode such that it advances the LV electrode distally while the RV electrode that remains secured in its position. The LV conductor lead is also shown extending distally as it slides out from the lead body and extends between the advanced LV electrode and secured RV electrode. FIG. 11D shows the pusher removed following proximal withdrawal, such that LV electrode remains extended distally from the RV electrode by a distance bridged by the LV conductor lead. This represents an illustrative configuration for implantation across a septum for example.

Various aspects of the embodiment shown and described by reference to FIGS. 10A-G, and by further reference to the physical embodiment demonstration shown in FIGS. 11A-D, may be modified by one of ordinary skill, and to the extent remaining consistent with the broad aspects contemplated hereunder and accomplishing one or more objectives set forth hereunder are considered to be within the scope of the present invention.

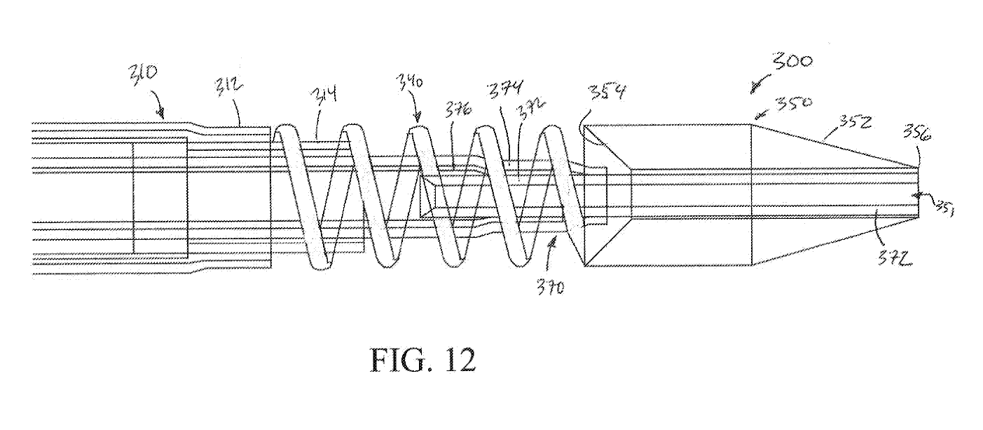

For example, FIG. 12 shows a biventricular pacing, single RV lead assembly 300 sharing many similarities with assembly 200 shown in FIGS. 10A-G, except with a modified LV electrode approach and that carries forward certain modifications to other related components. More specifically, in this present embodiment a body 310 includes a distal end portion 312 with an extended hypotube conductor 314 on which an RV electrode 340 of coiled screw type is secured, in similar fashion to the prior embodiment. Other related structures, though shown, are not herein described in detail though are also generally similar or otherwise slightly modified as appropriate to accommodate other modifications of the present embodiment. LV electrode 350, however, is different than LV electrode 250 in that this present configuration does not provide eccentric proximal taper or coupling for a pusher and the LV conductor lead 370. Rather, this approach is “coaxial” in nature. More specifically, LV electrode 350 includes a distal taper 352 but rather than a proximal taper it has a proximal inward taper 354. The distal tip 356 is not a sharpened point between two adjacent lumens. Rather, it is a distal port of a single central lumen 351 which is the only lumen in the electrode 350. Secured to the inner diameter of that central lumen is a distal electrically conductive tubular distal conductor 372 of the LV conductor lead assembly 370 that retains the central lumen 351 or bore coaxially within it. This tubular distal conductor 372 extends proximally to a proximal end that has a chamfered entrance to its inner diameter. Secured to an outer surface of the tubular distal conductor 372 is an outer tubular insulator 374 that also provides a robust flexible body to the LV conductor lead assembly 370. Also secured to the proximal end of the distal tubular conductor 372, but within outer insulator 374, is a conductor member 376 that extends along the body as insulated to ultimately couple to a coupler to engage an energy source for excitation of the LV electrode. The chamfered proximal entrance to distal tubular conductor 372 and into the inner bore or lumen 351 of LV electrode 350 is provided in order to engage a pusher (not shown) into and through the lumen until the pusher's enlarged proximal step-up region in outer diameter engages the chamfer (the chamfer assists in distal engagement to seat the pusher during assembly of the system for delivery, or subsequent to initial steps that may be for example over a guidewire prior to pushing the LV electrode to extend across a septum for implantation). It is believed that this present embodiment provides certain distinct benefits, including without limitation: availing more real estate by removing the need for two adjacent lumens into the LV electrode; and providing a coaxial arrangement between pusher and LV conductor lead presents more robust arrangement for hemostatic valving around the slideable engagement with the lead body, e.g. via a grommet or duckbill valve for example that can provide a single annular seal around both coaxially disposed components. Notwithstanding the latter benefit noted, a further valve or other form of closure to the inner lumen 351 following lead placement, to the extent it extends proximally with the LV conductor lead assembly 370 into the lead body 210, is also desired to prevent fluid ingress into the proximal assembly.

The embodiments presented hereunder that provide biventricular pacing via isolated RV and LV electrodes in an extendable transmuscular arrangement via a single RV lead assembly are considered highly beneficial in many circumstances of clinical utility and use. Many are presently contemplated, and others may become further developed by clinicians as patient management and sub-population basis becomes increasingly sophisticated over time and experience. One particular example is presented, without limitation, in the setting of proximal disease in the cardiac conduction system resulting in right and left bundle branch block (BBBB) along the septum, such that distal placement of right and left leads closely distal to the block(s) may provide optimally paced physiologic function.

Notwithstanding the benefits of these biventricular pacing approaches, however, it is also further appreciated that certain aspects presented by these particular embodiments providing transmuscular placement of LV electrodes for LV pacing may also be suitable modified and employed for only pacing the LV. In particular, several benefits have been reported in pacing the LV alone for certain patients. In this setting, providing the LV pacing may be accomplished via the transmuscular approaches for implanting the LV electrodes and stimulating the LV according to the present embodiments, either as presented by modified in their use for this alternative mode or purpose, or as suitable modified by one of ordinary skill to remove the RV pacing function and related structures.

In addition, in providing such assemblies for LV pacing alone via transmuscular/transseptal lead placement, certain further embodiments arise, including without limitation by reference to the following example.

### Example 1

The present example is premised at least in part upon the hypothesis that left ventricular pacing is useful for treatment of LV dysfunction in patients with a wide QRS. Disadvantages of LV pacing via the coronary sinus, the prevalent current adopted approach, include without limitation: inability to cannulate the CS; high capture thresholds; phrenic nerve stimulation; pacing sites limited by tributary anatomy; and epicardial-endocardial activation sequence. Accordingly, it is believed that pacing the LV via the endocardial wall on the left side of the interventricular septum via transseptal delivery from the RV would present significant advantage for LV pacing in many cases, and present improved Hemodynamics versus RV pacing, and/or versus LV pacing via coronary sinus LV lead placement. Moreover, biventricular pacing is believed to benefit from LV stimulation in the sub-endocardial septal location via electrodes delivered in the transseptal approach from the RV.

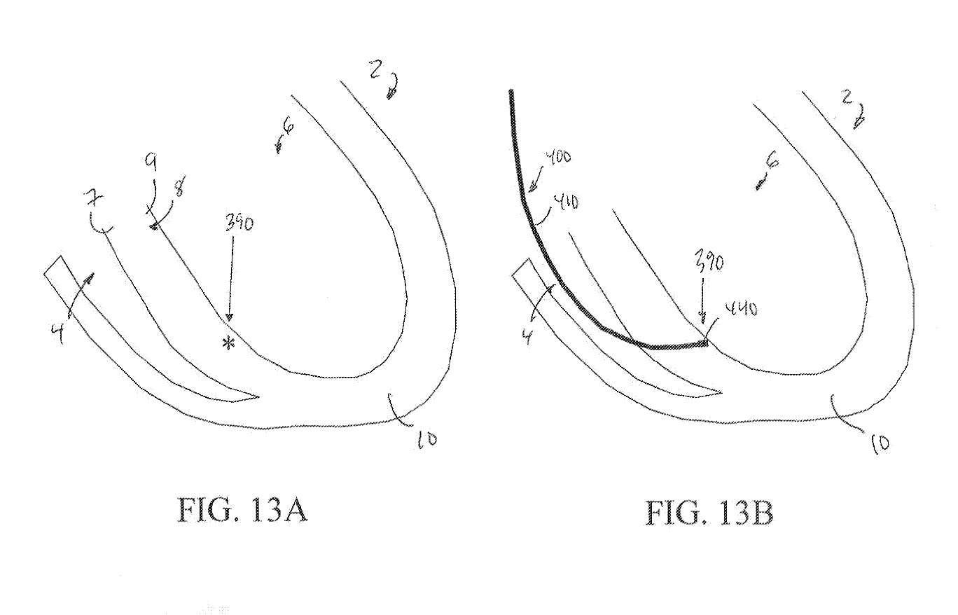

FIGS. 13A-24 show various aspects of the present disclosure related to LV pacing via transseptal LV electrode delivery from the RV and implantation along the LV endocardium. In general context, FIGS. 13A-B illustrate a generally schematic approach for this purpose. FIG. 13A illustrates a desired location 390 for LV pacing via transseptal placement from an RV lead. This location is subendocardial on LV side 9 of the septum 8, and may be, for example, distal to a left bundle branch block or “LBBB”. In this setting, pacing the left ventricle is considered beneficial, and may be accompanied by relatively healthy RV conduction system not requiring pacing, or by an also diseased RV conduction system that is not directly paced, or the latter but that is paced elsewhere. FIG. 13B shows a schematic representation of an RV lead system 400 with a lead body 410 extending across a septum 8 to an LV electrode 440 at its distal tip that paces the LV from the endocardial region 390 shown in FIG. 13A.

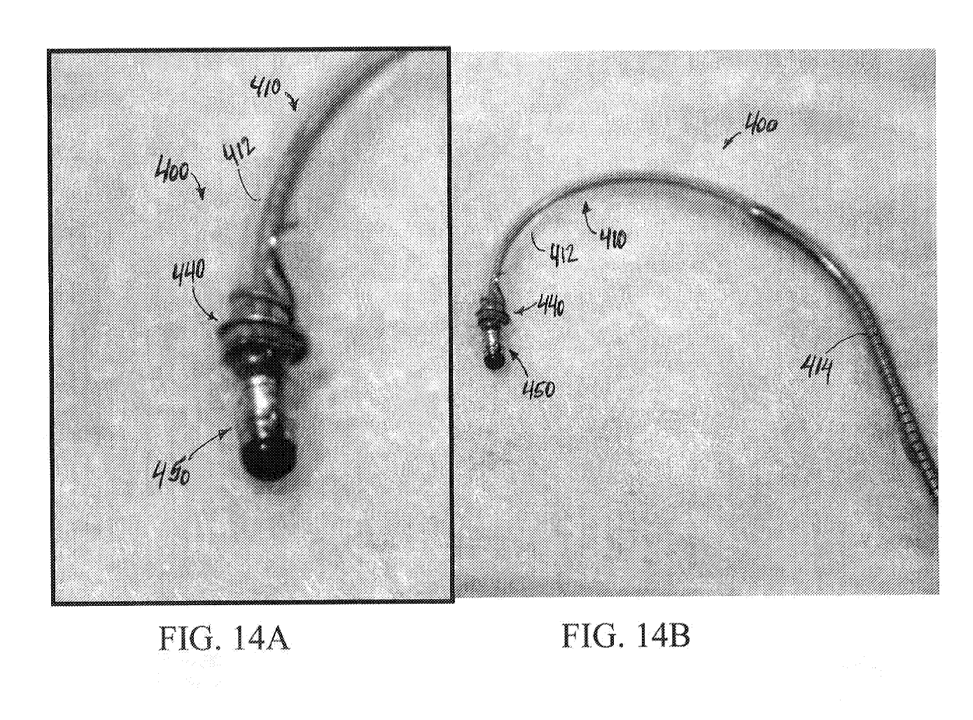

FIGS. 14A-B show certain detail of a highly beneficial trans-septal, LV stimulation lead embodiment according to the present example which has been developed and utilized in certain experiments, and thus presented hereunder by way of this Example 1 as follows.

FIGS. 14A-B show a photograph of a transseptal LV pacing lead 400 that was constructed according to the experiments conducted under the current Example 1. Lead 400 includes a lead body 410 with a distal end portion that includes an outer helical screw 440 winding around the body 410 proximally adjacent to an LV electrode 450 on a distal tip thereof. A proximal portion 414 of body 410 is shown in FIG. 14B for illustration purpose. This lead assembly 400 was constructed as shown by bonding the helical screw 440 onto a commercially available and widely adopted Medtronic® “BiPolar LV Pacing Lead”, model number 4194, in the relative location shown along the lead in FIGS. 14A-B. This lead includes a valve at its tip and is generally designed for intended use for over the wire coronary sinus placement for LV pacing. However, as explained in further detail hereunder, the present embodiments modify the device and use for transseptal delivery to a subendocardial location within the LV side of the septum for more proximal septal activation of LV pacing.

According to the experiment performed, 6 mongrel dogs were placed under general anesthesia and implants were performed in a sterile fashion on IV heparin. All animals underwent radiofrequency ablation of the atrio-ventricular junction and placement of standard pacing leads (e.g. right atrium or “RA”, right ventricle or “RVA”, and coronary sinus or “CS” for LV pacing). Acute hemodynamics were recorded during DDD pacing using a Millar® high fidelity pressure micromanometer in the left ventricle. The standard baseline measurements taken from these standard approaches were compared against approaches with the present transmuscular LV septal lead placement embodiment.

Placement of the transseptal LV leads was accomplished as follows. A transseptal sheath and needle assembly is delivered via the jugular vein, superior vena cava, right atrium, across the tricuspid valve, and into the RV and against the RV side of the interventricular septum. The needle is then pushed to advance across the septum until it breaches into the LV, as confirmed by contrast injection. The transseptal sheath is advanced over the needle and across the septum. The needle is withdrawn and replaced with a guidewire that is advanced through the sheath and into the LV, and may be further advanced through the aortic valve and into the aorta. The transseptal LV pacing lead, such as shown in FIGS. 14A-B, is advanced over the guidewire engaged through the inner guidewire lumen and via the valved tip, and along the wire across the septum and into the LV. The sheath may be pulled back at this point, or another point subsequently. The LV pacing lead is then pulled proximally back toward the septum. If the sheath is then pulled back, the outer screw surface 440 encounters the septum at the LV side of the through puncture. At this point continued withdrawal under rotation screws the lead backwards from the LV into the septum until the electrode arrives at the subendocardial location desired. Desired location can be determined by testing capture threshold at various points during the retraction (e.g. low capture threshold represents desired sub-endocardial placement).

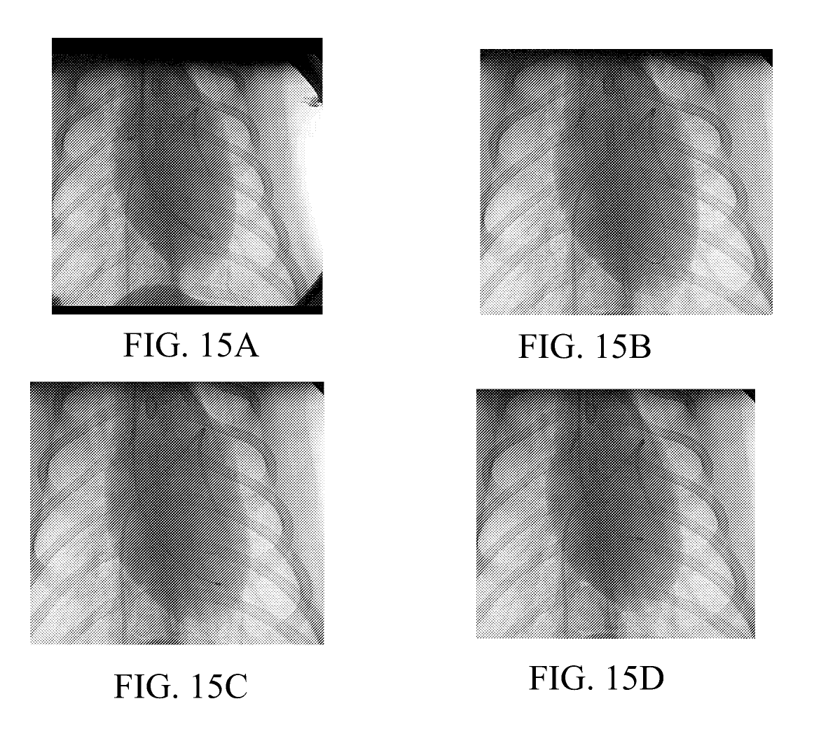

FIGS. 15A-D show captured fluoroscopic images of one exemplary treated subject for further illustration of this experiment performed, and believed to represent a clinically relevant approach. More specifically, FIG. 15A shows a transseptal needle positioned across the ventricular septum and with its tip in the LV, as confirmed by a small contrast injection. FIG. 15B shows a guidewire subsequently positioned down the superior vena cava, across the right atrium, into the RV, extending transseptally into the LV, and out of the LV into the aorta and beyond. This distal purchase of the guidewire allows for robust seating and operation of the LV lead system through the septum along a stiff portion of the guidewire, though not necessarily required to accomplish this specific arrangement in many circumstances. FIG. 15C shows an LV lead according to the present embodiment after being positioned transseptally over the guidewire and secured into the subendocardium via withdrawal and torsioned screwing action backward from the LV. FIG. 15D shows a more superior placement of the LV lead at a location along the septum more proximal in the LV conduction bundle.

According to this experimental approach, 9F and/or 11F transseptal sheaths were used. The lead diameter may be about 6F at its tip, and about 4F along its proximal shaft. While the present experiment used these equipments according to their ready availability, it is contemplated that in a custom system optimized for this intended use, smaller diameter tools are considered to be both suitable and desired. Moreover, certain components are believed to be unnecessary in certain circumstances. For example, in many cases a transseptal sheath may not be required at all, and the guidewire may be positioned across the septum (as well as the over the wire LV lead) without it.

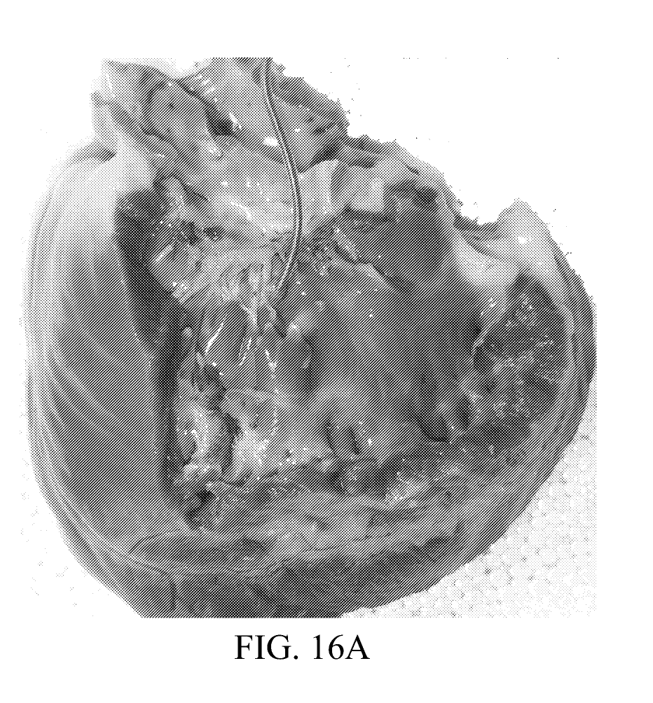

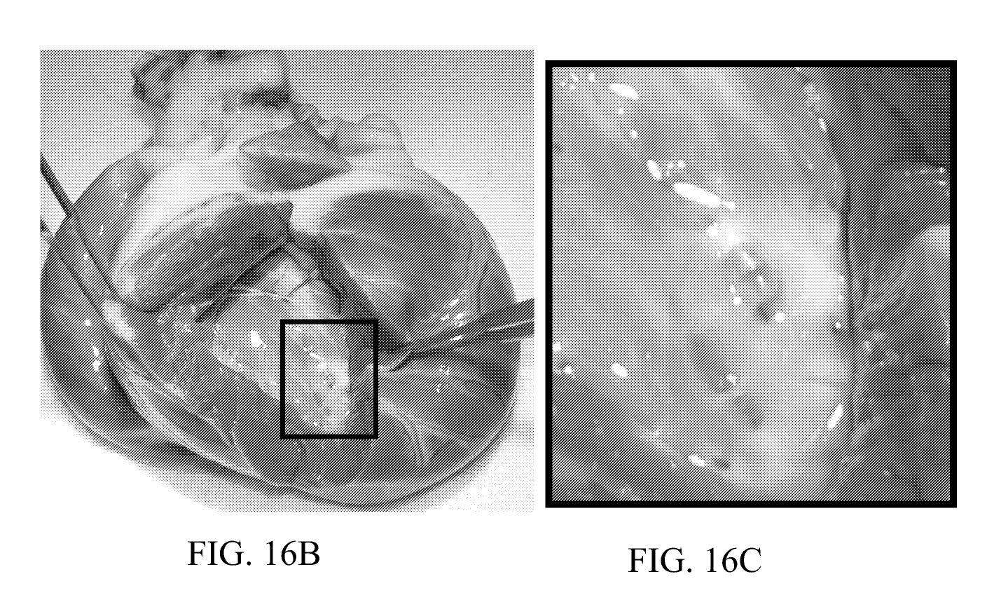

FIG. 16A shows a post mortem photograph of a lead positioned as just described, as seen from the RV side of the septum. FIGS. 16B-C show the same lead, from the LV side of the septum. As noted in the photographic FIGS. variously regarding this present experiment, the transseptal delivery of the LV lead is accomplished at an oblique angle, via the basal-to-apical orientation of the transseptal delivery modalities entering the RV from the right atrium. As a result, the LV electrode on the distal tip of the lead is more apical along the septum as seen in FIGS. 16B-C than the introduction to the septum on the RV side per the FIG. 16A view. In a setting of biventricular pacing adopting this approach to a second RV pacing electrode, according to a still further embodiment of the present disclosure, the RV electrode may thus have a more basal location and more proximal in the right side conduction system than the LV electrode and vis-à-vis the left side conduction system.

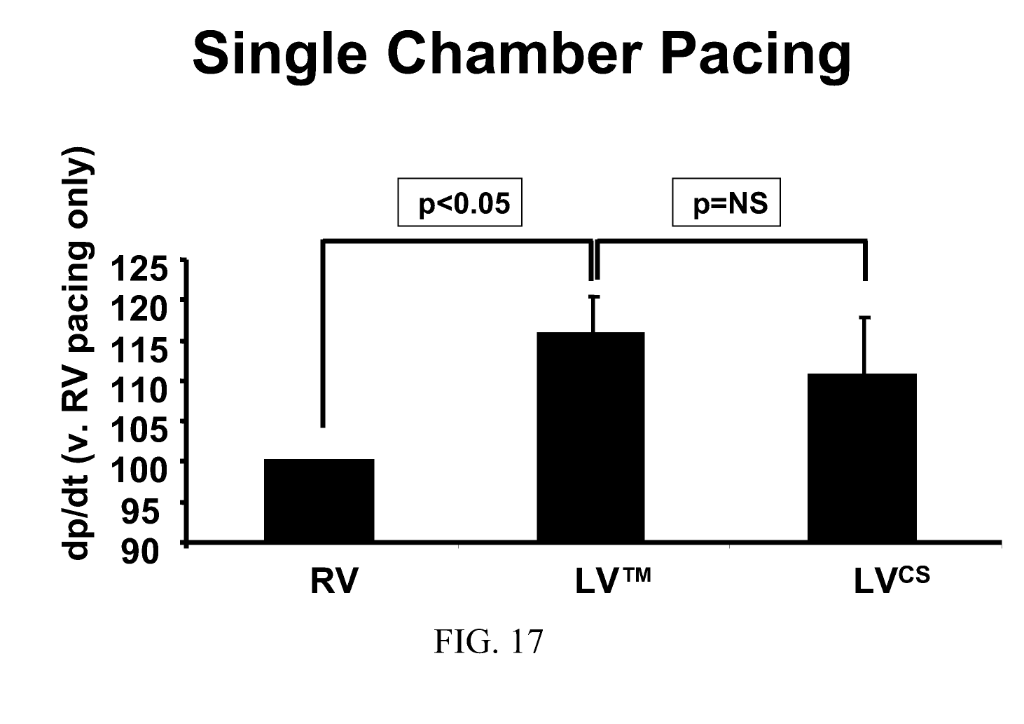

Results of the experiment revealed a mean acute threshold of 0.69+/−0.3V at 0.5 ms, and no echo evidence of VSD or LV thrombus. Autopsy was performed after 4 weeks of chronic pacing, and demonstrated the transmuscularly delivered LV pacing lead was endothelialized with threshold at 0.75V at 0.5 ms. Further comparison results between the approach of this present embodiment and other pacing modalities with respect to cardiac pumping efficiency are further described as follows by reference to graphical data shown in FIGS. 17-24.

FIG. 17 reflects change in pressure over time as a measure of contractility and pumping efficiency, expressed as a percentage of data resulting from RV pacing alone as normalizing basis. RV pacing alone is compared against transmuscular LV pacing alone (LVTM) and coronary sinus based LV pacing alone (LVCS). As shown in the graph, LVTM alone was significantly better than RV alone, and revealed evidence of some improvement over LVCS pacing alone (although without statistical significance drawn between the latter comparison from this limited sample size study).

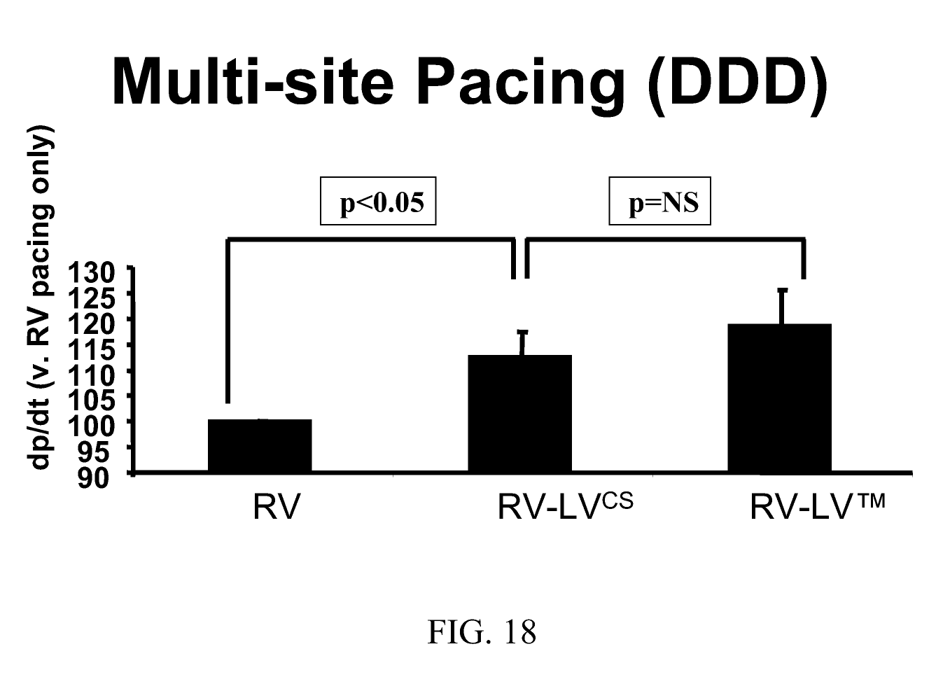

FIG. 18 show a similar graph on similar metrics, but comparing RV pacing alone against biventricular pacing via RV pacing as combined with either LVCS pacing or LVTM pacing. As shown in this graph, RV pacing alone was statistically inferior in contractility result versus RV-LVCS which was not statistically different than RV-LVTM biventricular pacing (though again the mean data reflects trend evidence of still further improvement from RV-LVTM versus RV-LVCS biventricular pacing).

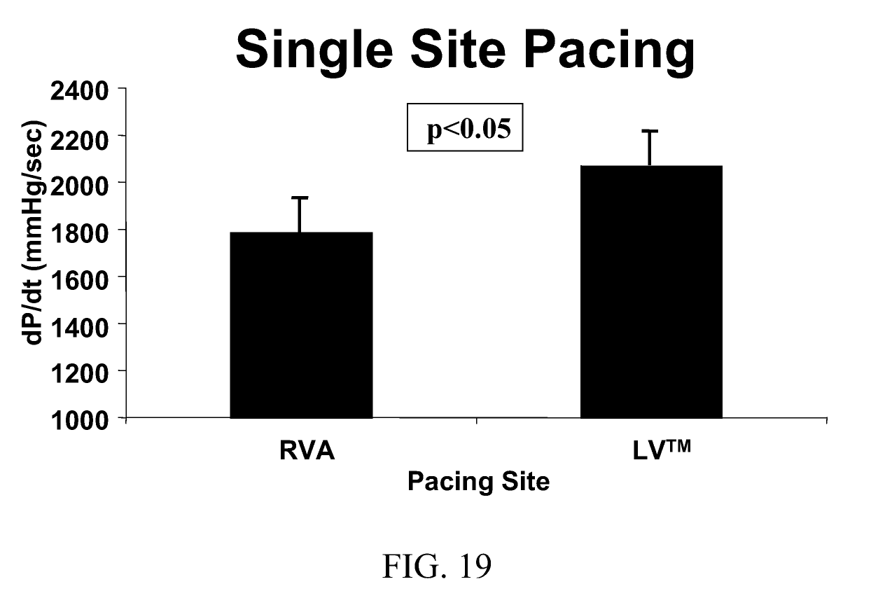

FIG. 19 shows similar comparison for single site pacing between RV alone and LVTM alone as reflected in the data in FIG. 17, but without normalization to the RV alone data and thus reflecting actual data for dP/dt under the comparison conditions. As shown, the raw data comparison reveals statistically significant improvement from the LVTM approach versus RV alone.

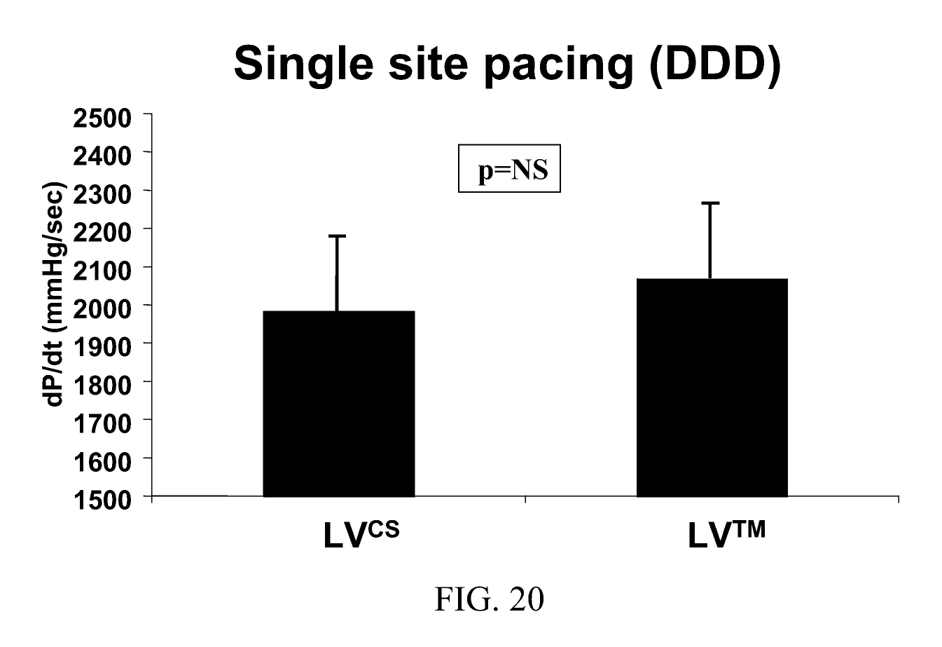

Similarly, FIG. 20 compares single site pacing between LVCS and LVTM alone conditions, per raw dP/dt datat results. The mean data for LVTM appears to trend slightly improved versus LVCS, though again no statistical significance per this data sampling size.

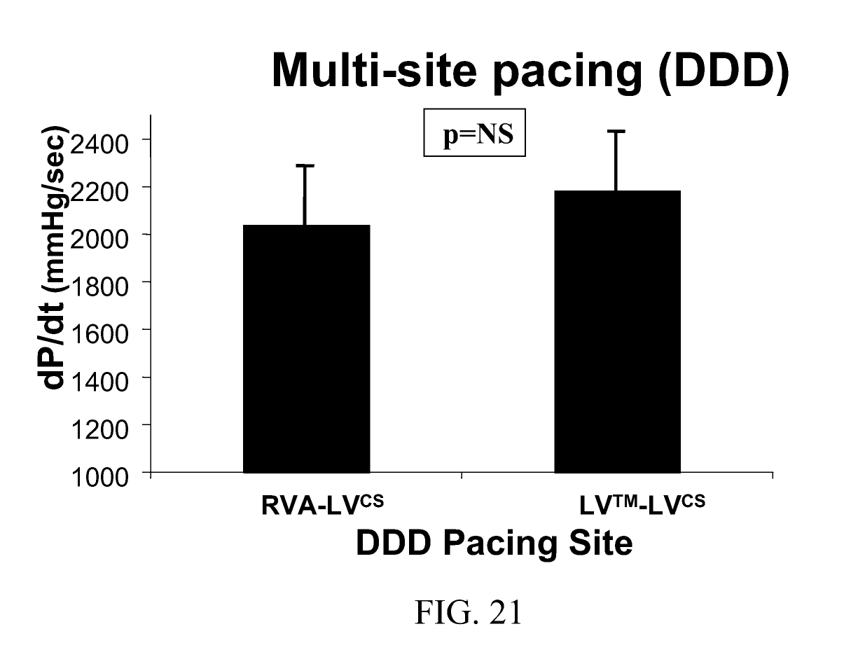

In still further comparison, multisite pacing was compared in FIG. 21 between RV-LVCS and LVTM-LVCS pacing. Here, the RV-LVCS represents conventional biventricular pacing, and reflects the generally accepted norm in clinical use for such biventricular pacing. In contrast, the LVTM-LVCS approach is multisite, but single chamber in the LV. This observes whether two initiations on the LV side, one at the septum and one at the free wall (via the LVCS lead) improves over the standard dual chamber biventricular pacing modality. No statistical significance was found, though the LVTM group mean was in fact again higher.

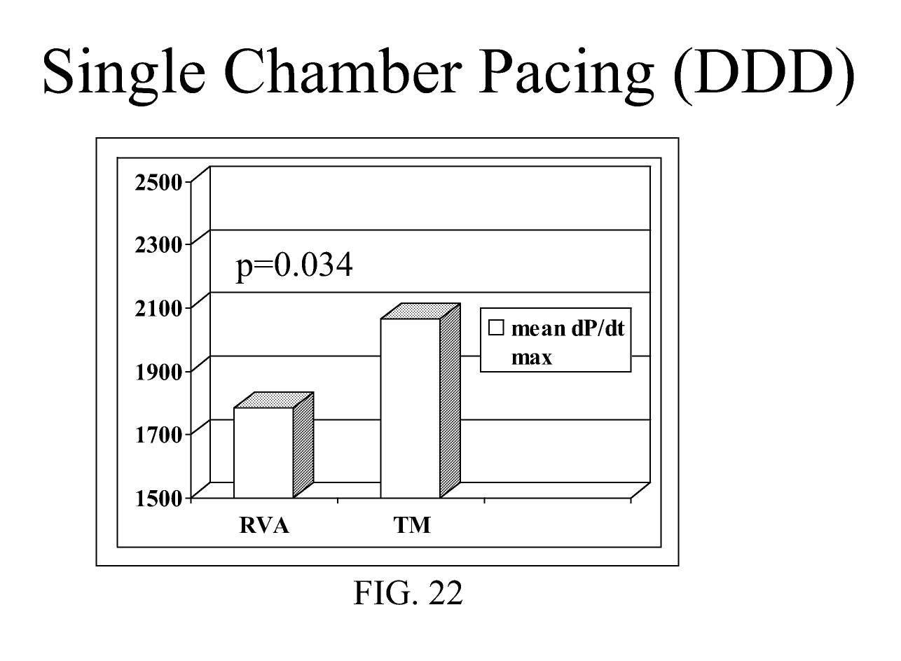

In slightly modified data analysis, FIGS. 22-24 show graphical results of data reflected above, but when comparing the dP/dt performance based upon peak measurements found in each case (versus mean data from each treatment). Differences (if any) in peak contractility is thus observed.

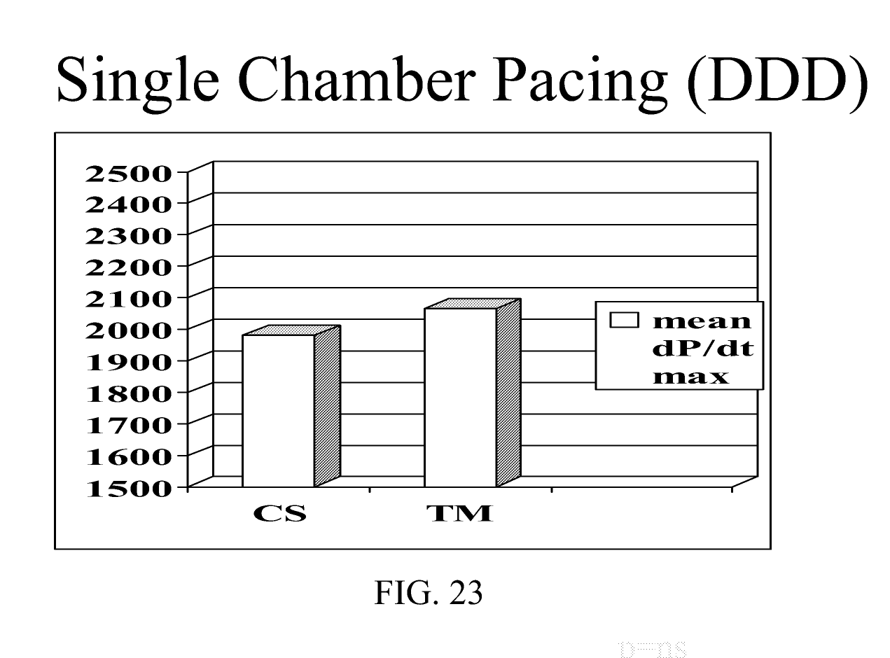

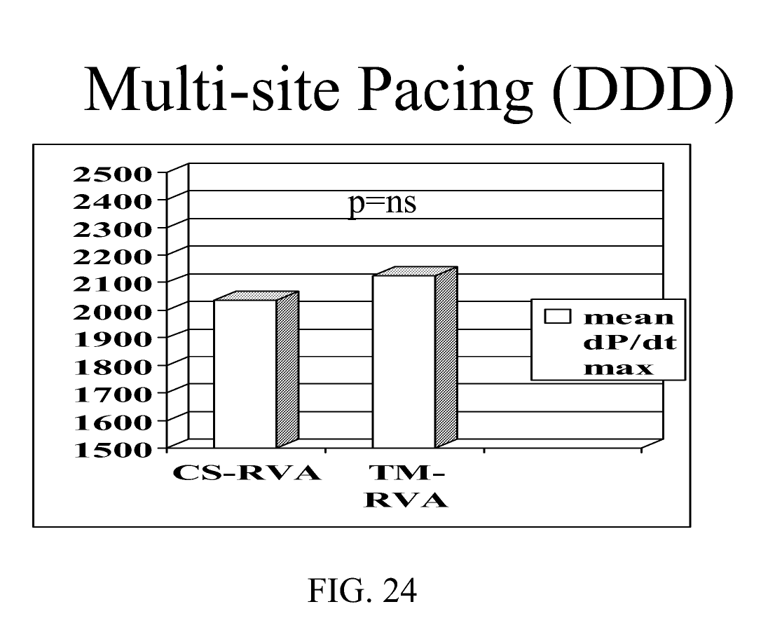

As shown in FIG. 22, transmuscular (TM) approach to LV pacing is significantly improved over RV alone, with p-value of 0.034, in single site pacing comparison. FIG. 23 shows the TM approach again not significantly different than the CS approach, though the data was higher in the TM group of the present embodiment. In FIG. 24, multisite biventricular pacing in TM-RV approach is again higher than CS-RV combination approach, though again without statistical significance in this limited number of samples.

According to the foregoing, LVTM approach revealed significant improvement over other approaches to pacing. Even where significance was not found statistically, in every comparison presented in the present Figures, the LVTM approach represented the highest performance data in the results, either in single chamber or dual chamber (or otherwise biventricular) pacing. Accordingly, the benefit and utility of transmuscular LV septal pacing is confirmed by the present experiment. Moreover, combination of the LV lead and placement of the present embodiment with the dual electrode, single RV lead embodiments elsewhere herein presented, is believed to present still further prospective benefit in many settings. The transseptal lead presented is also particularly adapted for transmuscular delivery and placement of the lead, and thus may be further included in one or more of the biventricular assemblies elsewhere shown and described hereunder, as modified appropriately according to one of ordinary skill.

The following Published US patent applications is herein incorporated in its entirety by reference thereto: US 2004/0098075.

The following International PCT Patent Application Publications are herein incorporated in their entirety by reference thereto: WO 2000/222206 to Lee and WO 2004/045709 to Lee.

It is appreciated that various of the presently disclosed aspects provide for an LV stimulation lead of particular benefit. In this regard, it is to be appreciated that “stimulation” may encompass many specific forms and implementations to treat differing conditions with different desired results, including for example but without limitation the following: pacing, defibrillating, enhancing contractility, or combinations thereof (either simultaneously or under different modes of use).

It is also to be appreciated that the present disclosure features in particular lead designs and methods of use. However, as would be appreciated by one of ordinary skill in the art, such uses further include combinations in systems with energy sources. These may include for example actuators or energy sources to be coupled to and actuate stimulators (e.g. electrodes) on the leads. Such actuators or sources include for example but without limitation the following: pacemakers, defibrillators, and other forms of stimulation sources such as to enhance contractility. These may be implantable sources, or may energize electrodes or other stimulators remotely such as via inductive fields, ultrasound, etc. Telemetry and “smart” software may be included in such systems. The further inclusion of one or more such further components in combination with the lead embodiments herein described are considered further aspects hereunder. The combination of components may also be provided pre-packaged or bundled together in such overall systems or assembled portions thereof, though providing the leads themselves is considered of independent benefit even though such may principally arise by further combination with other such components in a completed stimulation system.

The terms “electrodes” and “stimulators” are herein used variously throughout this disclosure, and unless otherwise further limited or defined by more specific description are generally intended to cover electrical stimulation, though other forms of stimulation such as ultrasonic etc. may be substituted without departing from the various broad aspects herein contemplated. Nonetheless, the specific stimulation via electrical energy is considered of particular widespread benefit in many available uses.

Where “dual electrode” assemblies are described herein, or like terms, in context of two excitation or stimulation electrodes, it is to be appreciated that in cardiac stimulation a circuit is to be completed to include such stimulation electrodes, the energy source, and some return or reference electrode. To the extent of dual excitation electrodes in single lead systems, such may include a single reference electrode shared between the two excitation electrodes, or two separate reference electrodes. The reference electrodes are generally positioned at least slightly remotely from the excitation electrodes in order to avoid too much current density in the area, and of sufficient surface area as well to avoid unwanted hightened levels of current density. Such reference electrode(s) may be proximally located along a lead shaft, for example, or otherwise provided as appropriate to one of ordinary skill.

The various detailed descriptions of the specific embodiments may be further combined in many differing iterations, and other improvements or modifications may be made that are either equivalent to the structures and methods described or are obvious to one of ordinary skill in the art, without departing from the scope of the invention. The illustrative examples therefore are not intended to be limiting to the scope of the claims below, or with respect to the Summary of the Invention, unless such limitation is specifically indicated.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A cardiac stimulation system, comprising:
an RV lead body with a proximal end portion and a distal end portion terminating in a distal tip;
a tissue fixation member at a first location along the distal end portion and proximally of the distal tip;
a left ventricle (LV) stimulator at a second location along the distal end portion;
wherein the second location is distally adjacent the first location such that the tissue fixation member when affixed to tissue is adapted to secure the cardiac stimulator at a fixed position within a ventricular wall;
wherein the tissue fixation member comprises a ridged outer surface at the first location along the distal end portion;
wherein the (LV) stimulator is configured to be positioned transmuscularly from a right ventricle (RV) chamber, across an interventricular septum, and to a location along a left side of the septum, and is configured to be actuated so as to couple energy to tissue so as to stimulate principally the LV conduction system from the location; and
wherein the LV stimulator is adapted to be coupled to an actuator at a remote location from the fixed position and to be actuated by the actuator in a stimulation mode of operation to stimulate cardiac tissue at the fixed position.

2-46. (canceled)

47. The system of claim 1, wherein the ridged outer surface is helical along a length and is adapted to be screwed into tissue.

48. The system of claim 47, wherein the lead body comprises an internal passageway with a distal port at a distal tip of the distal end portion, and a hemostatic valve within the passageway at or adjacent to the distal port, and the valve is configured for a guidewire to be positioned therethrough in an open configuration but that is configured to prevent fluid egress into the passageway in a closed configuration.

49. The system of claim 1, further comprising a transseptal delivery system configured to deliver the LV stimulator of the system across an interventricular septum at least for placement at a subendocardial location on a left side of the septum.

50. The system of claim 49, wherein the transseptal delivery system comprises a transseptal sheath.

51. The system of claim 49, wherein the transseptal delivery system comprises a transseptal needle.

52. The system of claim 1, wherein the system is configured for biventricular pacing in conjunction with a right ventricle (RV) stimulator.
